(19) United States
(12) Patent Application Publication
(10) Pub. No.: US 2026/0110700 A1
(43) Pub. Date: Apr. 23, 2026

(54) METHODS FOR REMOTE BLOOD COLLECTION, EXTRACTION AND ANALYSIS OF NEURO BIOMARKERS

(71) Applicants: Kaj BLENNOW, Kungsbacka (SE); Nicholas J. ASHTON, Billdal (SE); Laia MONTOLIU-GAYA, Göteborg (SE); John Henrik ZETTERBERG, Mölnlycke (SE); ALZPATH, INC., Carlsbad, CA (US)

(72) Inventors: ANDREAS JEROMIN, Alachua, FL (US); KAJ BLENNOW, KUNGSBACKA (SE); NICHOLAS J. ASHTON, BILLDAL (SE); LAIA MONTOLIU-GAYA, GÖTEBORG (SE); JOHN HENRIK ZETTERBERG, MÖLNLYCKE (SE)

(21) Appl. No.: 19/425,343

(22) Filed: Dec. 18, 2025

Related U.S. Application Data
(63) Continuation of application No. PCT/US24/37834, filed on Jul. 12, 2024.
(60) Provisional application No. 63/513,815, filed on Jul. 14, 2023.

(51) Int. Cl. G01N 33/68; G01N 1/34; G01N 33/50; G01N 33/53

(52) U.S. Cl. CPC G01N 33/6896; G01N 1/34; G01N 33/5002; G01N 33/53; G01N 2333/4709; G01N 2800/2814; G01N 2800/50; G01N 2800/60

(57) ABSTRACT

Provided are methods and kits for blood sample collection, protein extraction, and analysis of neuro biomarkers obtained from blood samples on dried blood drop cards.

### CROSS-REFERENCE TO RELATED APPLICATIONS

This application is a continuation of International Patent Application No. PCT/US24/37834, filed Jul. 12, 2024, which claims the benefit of U.S. Provisional Patent Application No. 63/513,815, filed on Jul. 14, 2023 which is incorporated by reference herein in its entirety.

### BACKGROUND

An accurate diagnosis of neurodegenerative disorders such as Alzheimer's disease (AD) and other tauopathies, as well as conditions such as traumatic brain injury (TBI) is important for prognostication and for the enablement of disease-modifying treatments. Neurofilament light (NfL), glial fibrillary acidic protein (GFAP) and phosphorylated tau are leading biomarkers for neurodegeneration, glial activation and AD pathology, respectively, that are detectable in blood. Yet, current recommendations utilize rapid centrifugation and ultra-low temperature storage after venipuncture collection, which can be inconvenient for sample collection and analysis of the neuro biomarkers. There is an unmet need for accessible methods to collect blood samples and analyze these neuro biomarkers.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### SUMMARY

Provided herein are methods and compositions for analysis of neuro biomarkers using blood dropped on dried plasma spot card devices.

In one aspect, the present disclosure provides a method for determining protein levels of one or more biomarkers in a biological sample, the method comprising: a) obtaining the biological sample, wherein the biological sample is a capillary blood sample derived from a subject; b) extracting proteins from the biological sample using a protein extraction reagent to obtain a protein-enriched extracted sample; c) performing an assay on the protein-enriched extracted sample, wherein the assay comprises determining a detected protein level of at least one of biomarkers: i) phosphorylated Tau-181 (pTau-181), ii) phosphorylated Tau-217 (pTau-217), iii) glial fibrillary acidic protein (GFAP), iv) neurofilament light chain (NFL), v) phosphorylated Tau-231 (pTau-231), vi) phosphorylated Tau-212 (pTau-212), vii) phosphorylated Tau-212+217 (pTau-217+), viii) brain-derived tau (BD-Tau), ix) 42-amino acid β amyloid (Aβ42) peptide, or x) 40-amino acid 1 amyloid (Aβ40) peptide.

In some embodiments, the protein extraction reagent comprises a buffered solution comprising tris(hydroxymethyl)aminomethane (Tris), sodium chloride (NaCl), bovine serum albumin (BSA), and a polysorbate nonionic surfactant. In some embodiments, the protein extraction reagent comprises a buffered solution comprising phosphate buffered saline (PBS) comprising phosphate, NaCl, and potassium chloride (KCl). In some embodiments, the buffered solution further comprises a polysorbate nonionic surfactant. In some embodiments, the buffered solution further comprises EDTA. In some embodiments, the buffered solution further comprises BSA. In some embodiments, the buffered solution further comprises magnesium chloride (MgCl₂), dextrose, and urea. In some embodiments, the buffered solution further comprises a TRU Block™ immunoassay blocker. In some embodiments, the protein extraction reagent further comprises a preservative. In some embodiments, the preservative comprises ProClin™ 300. In some embodiments, the polysorbate nonionic surfactant comprises either polyethylene glycol sorbitan monolaurate (Tween 20) or polyethylene glycol sorbitan monooleate (Tween 80). In some embodiments, the polysorbate nonionic surfactant comprises Tween 20. In some embodiments, the buffered solution has a pH between about 7.0-9.0. In some embodiments, the buffered solution has a pH of about 7.4. In some embodiments, the buffered solution has a pH of about 9.0. In some embodiments, the TRU Block™ immunoassay blocker of the buffered solution is at concentration of about 1%. In some embodiments, the protein extraction reagent comprises about 20 mM Tris, about 137 mM NaCl, about 1% BSA, about 0.05% ProClin™ 300, about 0.10% Tween 20, and about 5 mM EDTA.

In some embodiments, the step b) further comprises incubating the biological samples for 30 minutes. In some embodiments, the step b) further comprises transferring the biological sample to a quantification plate. In some embodiments, the biological sample is transferred to the quantification plate via centrifugation through a filter plate.

In some embodiments, the assay in step c) comprises an immunoassay. In some embodiments, the immunoassay comprises Western blot analysis, dot blot analysis, flow cytometry-based immunoassay, enzyme-linked immunosorbent assay (ELISA), lateral flow immunoassay, radioimmunoassay (RIA), competition immunoassay, dual antibody sandwich assay, chemiluminescent assay, bioluminescent assay, fluorescent assay, or agglutination assay.

In some embodiments, the immunoassay comprises: i) single-molecule immunosorbent assay (Simoa®), ii) Meso Scale Discovery high performance electrochemiluminescence (MSD) assay, iii) Lumipulse® assay, iv) Elecsys® assay, v) Ella® Immunoassay (ProteinSimple, Bio-Techne), vi) ARCHITECT® Immunoassay (Abbott), vii) Atellica® Immunoassay (Siemens Healthineers), viii) ARGO™ HT Immunoassay (Alamar), or ix) Olink® immunoassay platform proximity extension assay system (Thermo Fisher Scientific). In some embodiments, the immunoassay comprises i) Simoa®. In some embodiments, the assay comprises use of the Olink® immunoassay platform proximity extension assay system (Thermo Fisher Scientific. In some embodiments, the assay comprises mass spectrometry.

In some embodiments, the capillary blood sample comprises a plurality of drops of blood. In some embodiments, the capillary blood sample comprises a single drop of blood. In some embodiments, the capillary blood sample has been deposited on a blood sample collection card to create a spotted blood sample. In some embodiments, the volume of the spotted blood sample is less than about 100 μL. In some embodiments, the blood sample collection card comprises a plasma separation card. In some embodiments, the plasma separation card generates cell-free plasma from the spotted blood sample to use as the biological sample in step a). In some embodiments, cell-free plasma is collected on at least two plasma separation membranes. In some embodiments, at least one of the at least two plasma separation membrane undergoes protein extraction in step b) using a protein extraction reagent comprising a buffered solution comprising Tris, NaCl, BSA, and a polysorbate nonionic surfactant; wherein at least one of biomarkers: i) pTau-181, ii) pTau-217, v) pTau-231, vi) pTau-212, vii) pTau-217+, or viii) BD-Tau is assayed to determine the detected protein level.

In some embodiments, at least one of the at least two plasma separation membrane undergoes protein extraction in step b) using a protein extraction reagent comprising a buffered solution comprising Tris, NaCl, BSA, and a polysorbate nonionic surfactant; wherein at least one of biomarkers: i) pTau-181, ii) pTau-217, or v) pTau-231 is assayed to determine the detected protein level. In some embodiments, ii) pTau-217 is assayed to determine the detected protein level. In some embodiments, at least two of i) pTau-181, ii) pTau-217, v) pTau-231, vi) pTau-212, vii) pTau-217+, or viii) BD-Tau are assayed to determine the detected protein level.

In some embodiments, the protein extraction reagent further comprises EDTA.

In some embodiments, at least one of the at least two plasma separation membrane undergoes protein extraction in step b) using a protein extraction reagent comprising a buffered solution comprising PBS; wherein at least one of biomarkers: iii) GFAP, iv) NFL, vi) Aβ42 peptide, or vii) Aβ40 peptide is assayed to determine the detected protein level. In some embodiments, the detected protein level of the biomarkers: ii) phosphorylated Tau-217 (pTau-217), iii) glial fibrillary acidic protein (GFAP), and iv) neurofilament light chain (NFL), are determined in step c). In some embodiments, the detected protein level of the biomarkers: i) phosphorylated Tau-181 (pTau-181), ii) phosphorylated Tau-217 (pTau-217), iii) glial fibrillary acidic protein (GFAP), and iv) neurofilament light chain (NFL), are determined in step c).

In some embodiments, the capillary blood sample is dried prior to step b). In some embodiments, the biological sample is stored for up to about two weeks at ambient temperature prior to extracting proteins in step b). In some embodiments, the biological sample is stored for up to about two months at about 4° C. prior to extracting proteins in step b). In some embodiments, the detected protein level of each biomarker tested derived from the capillary blood sample is significantly correlated with a determined protein level of each respective biomarker tested in plasma isolated from a venous blood sample from the subject.

In some embodiments, the detected protein level of each biomarker tested derived from the capillary blood sample is significantly correlated with a determined protein level of each respective biomarker tested in a cerebrospinal fluid (CSF) sample from the subject.

In some embodiments, the detected protein level of each biomarker tested is used to assess a risk of the subject for developing cognitive decline as a consequence of neurodegeneration, traumatic brain injury (regardless of severity, including mild traumatic brain injury or concussion), cardiac arrest (or other causes of brain hypoxia), a neuroinflammatory condition, or a combination thereof. In some embodiments, the detected protein level of each biomarker tested is used to pre-screen and identify if the subject has elevated risk for developing cognitive decline prior to the subject undergoing a clinical evaluation. In some embodiments, a risk of the subject developing cognitive decline is determined prior to the subject presenting one or more behavioral symptoms associated with cognitive decline. In some embodiments, the detected protein level of each biomarker tested is used as a component in diagnosis of a neurodegenerative condition. In some embodiments, wherein the detected protein level of each biomarker tested is determined in at least two biological samples, each taken from the subject at different times. In some embodiments, a change in detected protein level of each biomarker tested over time is used to monitor a progression of a neurodegenerative condition. In some embodiments, a change in detected protein level of each biomarker tested over time is used to assess a response to a treatment in the subject for a neurodegenerative condition.

In some embodiments, a change in detected protein level of each biomarker tested over time is used to assess an occurrence of an adverse effect during a treatment with an amyloid immunotherapy. In some embodiments, the adverse effect comprises amyloid related imaging abnormalities (ARIA). In some embodiments, the method further comprises sequentially monitoring an evolution of the adverse effect or sequentially monitoring a resolution of the adverse effect.

In some embodiments, the neurodegenerative condition comprises a tauopathy. In some embodiments, the tauopathy comprises Alzheimer's disease (AD) or Alzheimer's disease pathology or pathophysiology, preclinical AD, prodromal AD, AD dementia, Pick's disease, Niemann-Pick disease type C, frontotemporal dementia (FTD), frontotemporal lobar degeneration (FLD), chronic traumatic encephalopathy (CTE) or traumatic encephalopathy syndrome (TES), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, or a transgene-induced tauopathy. In some embodiments, the tauopathy comprises Alzheimer's disease.

In another aspect, the present disclosure provides a method of identifying if a subject is at risk for having or developing a neurodegenerative disease, the method comprising: a) obtaining a biological sample, wherein the biological sample is a blood sample derived from the subject; b) extracting proteins from the biological sample using a protein extraction reagent to obtain a protein-enriched extracted sample; c) performing an assay on the protein-enriched extracted sample, wherein the assay comprises determining a detected protein level of at least one of biomarkers: i) phosphorylated Tau-181 (pTau-181), ii) phosphorylated Tau-217 (pTau-217), iii) glial fibrillary acidic protein (GFAP), iv) neurofilament light chain (NFL), v) phosphorylated Tau-231 (pTau-231), vi) phosphorylated Tau-212 (pTau-212), vii) phosphorylated Tau-212+217 (pTau-217+), viii) brain-derived tau (BD-Tau), ix) 42-amino acid β amyloid (Aβ42) peptide, or x) 40-amino acid 3 amyloid (Aβ40) peptide; d) providing the detected levels of biomarkers to perform an evaluation step, wherein the evaluation step comprises comparing the provided detected levels of biomarkers in the protein-enriched extracted sample derived from the subject to threshold levels of biomarkers detected in a plurality of healthy subjects having normal cognitive function who have not been diagnosed with a neurodegenerative disease; and e) estimating a likelihood of the subject for having a neurodegenerative disease or developing a neurodegenerative disease, following completion of the evaluation step. In some embodiments, the blood sample is a capillary blood sample or a venous blood sample. In some embodiments, the blood sample is a capillary blood sample. In some embodiments, the blood sample is a venous blood sample. In some embodiments, the neurodegenerative disease is Alzheimer's disease. Detection of pathogenic change indicative of early Alzheimer's disease currently relies of neuroimaging techniques to localize and quantify deposited amyloid beta (AR) or cerebrospinal fluid sample analysis. Blood-based biomarkers detected in a capillary blood sample offer greater convenience and the possibility of remote sample collection. The inherent lower concentration of target analyte for neuro biomarkers in capillary blood samples, as compared to either CSF samples or venous blood samples has previously made reliably specific detection and quantification difficult. In some embodiments, the protein extraction reagent to obtain a protein-enriched extracted sample is a protein extraction reagent described herein. In some embodiments, the protein extraction reagent comprises less than about 25 mM Tris base, than less about 175 mM NaCl, less than about 2.5% BSA, and less than about 0.15% of a polysorbate nonionic surfactant. In some embodiments, the protein extraction reagent comprises 20 mM Tris base, 137 mM NaCl, 1% BSA, and 0.1% Tween-20. In some embodiments, the protein extraction reagent comprises 10 mM Tris base, 150 mM NaCl, 2% BSA, and 0.1% Tween-20. In some embodiments, the protein extraction reagent further comprises 5 mM EDTA. In some embodiments, the protein extraction reagent further comprises 10 μg/mL TRU Block™. In some embodiments, the protein extraction reagent has a pH of about 9.0. In some embodiments, the evaluation step in d) comprises observing an increased value of at least one of the biomarkers relative to the control (healthy subjects having normal cognitive function who have not been diagnosed with a neurodegenerative disease), wherein the increased value is indicative of the future development of Alzheimer's disease. In some embodiments, the detected levels of biomarkers provide in d) is elevated before the onset of clinical symptoms in the subject. In some embodiments, value of at least one of the detected levels of biomarkers correlates with the time to onset of one or more symptoms of Alzheimer's disease. In some embodiments, preferably the value of at least one of the biomarkers is elevated before the onset of clinical symptoms. In some embodiments, the method is capable of detecting the onset of Alzheimer's disease at least 1 month, at least 2 months, at least 3 months, at least 6 months, at least 9 months, at least one year, at least two years, preferably at least three years, more preferably four years, and most preferably at least five years, prior to the onset of one or more symptoms, preferably wherein the value of at least one of the biomarkers is elevated before the onset of clinical symptoms. In some embodiments, the method is repeated in order to monitor changes in capillary blood sample neuro biomarker levels in the subject and continue monitoring for changes that represent progression of a neurodegenerative disease.

In another aspect, the present disclosure provides a method for treating a neurodegenerative disease in a subject in need thereof, the method comprising: i) determining protein levels of one or more biomarkers in a biological sample by: a) obtaining a biological sample, wherein the biological sample is a blood sample derived from the subject; b) extracting proteins from the biological sample using a protein extraction reagent to obtain a protein-enriched extracted sample; and c) performing an assay on the protein-enriched extracted sample, wherein the assay comprises determining a detected protein level of at least one of biomarkers: i) phosphorylated Tau-181 (pTau-181), ii) phosphorylated Tau-217 (pTau-217), iii) glial fibrillary acidic protein (GFAP), iv) neurofilament light chain (NfL), v) phosphorylated Tau-231 (pTau-231), vi) phosphorylated Tau-212 (pTau-212), vii) phosphorylated Tau-212+217 (pTau-217+), viii) brain-derived tau (BD-Tau), ix) 42-amino acid β amyloid (Aβ42) peptide, or x) 40-amino acid 1 amyloid (Aβ40) peptide; and ii) administering a therapy to the subject. In some embodiments, the blood sample is a capillary blood sample or a venous blood sample. In some embodiments, the blood sample is a capillary blood sample. In some embodiments, the blood sample is a venous blood sample. In some embodiments, the neurodegenerative disease is Alzheimer's disease. In some embodiments, one or more symptoms of Alzheimer's disease are improved following administration of the therapy to the subject. In some embodiments, the one of more symptoms of Alzheimer's disease may include cognitive impairment, including language impairment, deficits in visual function, memory loss including worsening memory loss, impairment of short-term memory, increased anxiety, a sleeping disorder, personality changes in the subject, such as progressive passivity or marked agitation, decreased expressions of affection, depression and psychosis. In some instances, mild cognitive impairment (MCI) is an early symptom of Alzheimer's disease. In some embodiments, the methods comprises detecting the subject as being at risk of developing Alzheimer's disease by comparing determined protein levels of one or more biomarkers in a biological sample in i) to the amount or concentration of the one or more biomarkers in a control sample, wherein an increased value of at least one of the one or more biomarkers relative to the control is indicative of the future development of Alzheimer's disease. In some embodiments, the determined protein levels of one or more biomarkers in the biological sample in i) is elevated before the onset of clinical symptoms in the subject. In some embodiments, the amount of determined protein levels of one or more biomarkers in the biological sample in i) correlates with the time to onset of one or more symptoms of Alzheimer's disease. In some embodiments, preferably the amount of determined protein levels of one or more biomarkers is elevated before the onset of clinical symptoms. In some embodiments, the method is capable of detecting the onset of Alzheimer's disease at least 1 month, at least 2 months, at least 3 months, at least 6 months, at least 9 months, at least one year, at least two years, preferably at least three years, more preferably four years, and most preferably at least five years, prior to the onset of one or more symptoms, preferably wherein amount of determined protein levels of one or more biomarkers is elevated before the onset of clinical symptoms. In some embodiments, the method is repeated in order to monitor changes in capillary blood sample neuro biomarker levels in the subject.

In another aspect, the present disclosure provides kits comprising a protein extraction reagent and instructions for use. In some embodiments, the protein extraction reagent comprises less than about 25 mM Tris base, than less about 175 mM NaCl, less than about 2.5% BSA, and less than about 0.15% of a polysorbate nonionic surfactant. In some embodiments, the protein extraction reagent comprises 20 mM Tris base, 137 mM NaCl, 1% BSA, and 0.1% Tween-20. In some embodiments, the protein extraction reagent comprises 10 mM Tris base, 150 mM NaCl, 2% BSA, and 0.1% Tween-20. In some embodiments, the protein extraction reagent further comprises 5 mM EDTA. In some embodiments, the protein extraction reagent further comprises 10 μg/mL TRU Block™. In some embodiments, the protein extraction reagent further comprises 0.05% ProClin™ 300. In some embodiments, the instructions comprise instructions for a method of protein extraction and ultra-sensitive quantification described herein. In some embodiments, the kit further comprises a quantification plate. In some embodiments, the quantification plate is compatible for use in a Simoa® detection system, an Meso Scale Discovery (MDS) analysis system, a Lumipulse® immunoassay analysis system, an Elecsys Cobas® e801 immunohistochemistry analysis system, an Ella Automated Immunoassay System analysis system, an ARCHITECT Immunoassay System analysis system, an Atellica® Solution Immunoassay analysis system, an ARGO™ HT System Immunoassay analysis system, an Olink® immunoassay platform proximity extension assay system (Thermo Fisher Scientific), or a mass spectrometer. In some embodiments, the kit further comprises an antibody specific for detection of one or more selected neuro biomarkers. In some embodiments, the antibody specific for detection of one or more selected neuro biomarkers is a p-tau181 antibody, a p-tau212 antibody, a p-tau217 antibody, a p-tau217+ antibody, a p-tau-231 antibody, a total-tau antibody, a BD-tau antibody, a GFAP antibody, an NfL antibody, an Aβ40 antibody, an Aβ42 antibody, or an UCHL1 antibody.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1C show correlations of phosphorylated tau (p-tau) obtained from dry blood spot and obtained from blood plasma using various devices. FIG. 1A shows p-tau181 (Quanterix p-tau181/Qp-tau181) correlations. FIG. 1B shows p-tau217 correlations (using ALZpath p-tau217 antibody). FIG. 1C shows p-tau231 correlations (using UGOT p-tau231 antibody).

FIG. 2A-FIG. 2B show correlations of Glial Acidic Fibrillary Protein (GFAP) and Neurofilament Light (NFL) obtained from dry blood spot and obtained from blood plasma using various devices using 4-plex-E Quanterix assay kit. FIG. 2A shows a correlation graph of GFAP obtained from dry blood spot (venous) versus GFAP obtained from blood plasma using either a Noviplex or Capitainer DPS sampling card (Quanterix). FIG. 2B shows a correlation graph of NfL obtained from dry blood spot (venous) versus NfL obtained from blood plasma using either a Noviplex or Capitainer DPS sampling card (Quanterix).

FIG. 3A-FIG. 3B show measurement of Amyloid-β peptide (Aβ40 and Aβ42) obtained from dry blood spot and obtained from blood plasma using various devices using 4-plex-E Quanterix assay kit. FIG. 3A shows a correlation graph of Aβ42 obtained from dry blood spot (venous) versus Aβ42 obtained from blood plasma using either a Noviplex or Capitainer DPS sampling card. FIG. 3B shows a correlation graph of Aβ40 obtained from dry blood spot (venous) versus Aβ40 obtained from blood plasma using either a Noviplex or Capitainer DPS sampling card.

FIG. 4A-FIG. 4D show correlation of phosphorylated tau (p-tau): p-tau181 and p-tau217 obtained from either capillary card or venous card compared to p-tau181 and p-tau217 obtained from blood plasma. FIG. 4A shows a correlation graph of p-tau217 obtained from dry blood spot (capillary) using a DPS sampling card versus p-tau217 obtained from blood plasma. FIG. 4B shows a correlation graph of p-tau181 (capillary) obtained from dry blood spot using a DPS sampling card versus p-tau181 obtained from blood plasma. FIG. 4C shows a correlation graph of p-tau217 obtained from dry blood spot (venous) using a DPS sampling card versus p-tau217 obtained from blood plasma. FIG. 4D shows a correlation graph of p-tau181 (venous) obtained from dry blood spot using a DPS sampling card versus p-tau181 obtained from blood plasma.

FIG. 5A-FIG. 5D show correlation of Glial Acidic Fibrillary Protein (GFAP) and Neurofilament Light (NfL) obtained from capillary card or venous card compared to GFAP or NfL obtained from blood plasma. FIG. 5A shows a correlation graph of capillary DPS card versus plasma GFAP. FIG. 5B shows a correlation graph of venous DPS card versus plasma GFAP. FIG. 5C shows a correlation graph of capillary DPS card versus plasma NfL.

FIG. 5D shows a correlation graph of venous DPS card versus plasma NfL.

FIG. 6A-FIG. 6C show higher level of NfL, GFAP, and p-tau181 biomarkers obtained from subjects with amyloid positive status when compared to the level of biomarkers obtained from the subjects with amyloid negative status. Biomarkers were obtained using dried blood drop cards. Status of amyloid protein from cerebrospinal fluid (CSF) is an indicator of Alzheimer's disease (AD) pathology.

FIG. 7 shows a summary of correlations between biomarkers in different fluid samples.

FIG. 8A-FIG. 8B show relationships of plasma biomarker concentrations to finger-prick and venous dried plasma spot measurements.

FIGS. 9A-9B shows variation in biomarker concentration by specimen and by participant group. FIG. 9A shows Group-wise concentrations of GFAP (top row) and NfL (lower row). FIG. 9B shows Forest plot showing % change in concentration of biomarkers by group (compared with healthy controls) in regression model for log(biomarker concentration.

FIG. 10 shows an overview of the suggested workflow from remote collection to laboratory analysis to test for biomarkers of neurodegenerative diseases, e.g., tauopathy.

FIG. 11 shows a graph and significant correlation of capillary p-tau217 levels compared to venous p-tau217 levels.

FIG. 12A-FIG. 12C show the sensitivity and specificity of p-tau217 Simoa® immunoassay results from capillary blood samples in accurately determining Aβ-positive or Aβ-negative status. FIG. 12A shows a graph of p-tau217 levels from plasma samples in Aβ-negative individuals (A−) and in Aβ-positive individuals (A+). FIG. 12B shows a graph of p-tau217 levels from capillary DPS card collected samples in Aβ-negative individuals (A−) and in Aβ-positive individuals (A+). FIG. 12C shows a receiver operating characteristic (ROC) graph indicating high sensitivity and specificity for Simoa® detected p-tau217 levels in capillary blood samples collected from the subjects.

FIG. 13 shows a graph and significant correlation of capillary p-tau217+ levels compared to venous p-tau217+ levels using Simoa® detection and a Janssen p-tau217+ antibody.

FIG. 14A-FIG. 14C show that p-tau181 Simoa® immunoassay results from capillary blood samples accurately determine Aβ-positive or Aβ-negative status. FIG. 14A shows a graph and significant correlation of capillary p-tau181 levels compared to venous p-tau181 levels. FIG. 14B shows a graph of p-tau181 levels from capillary DPS card samples in Ap-negative individuals (A−) and in Aβ-positive individuals (A+). FIG. 14C shows a graph of p-tau181 levels from venous plasma samples in Aβ-negative individuals (A−) and in Ap-positive individuals (A+).

FIG. 15A-FIG. 15C show correlations of capillary blood sample to venous blood sample levels of p-tau biomarkers using Simoa® quantification with University of Gotherberg (UGOT) antibodies. FIG. 15A shows a graph and significant correlation of capillary p-tau181 DPS levels compared to venous plasma p-tau181 levels. FIG. 15B shows a graph and significant correlation of capillary p-tau217 DPS levels compared to venous plasma p-tau217 levels. FIG. 15C shows a graph and significant correlation of capillary p-tau231 DPS levels compared to venous plasma p-tau231 levels.

FIG. 16A-FIG. 16B show correlations of capillary blood sample to venous blood sample levels of GFAP and NfL biomarkers using Simoa® quantification. FIG. 16A shows a graph and significant correlation of capillary GFAP levels compared to venous GFAP levels.

FIG. 16B shows a graph and significant correlation of capillary NfL levels compared to venous NfL levels.

FIG. 17A-FIG. 17D show results from monitoring biomarker levels following acute TBI (e.g., concussion). FIG. 17A shows a graph of GFAP levels from capillary blood samples from a subject between Day 0-Day 10 following a concussion. FIG. 17B shows a graph of UCHL-1 levels from capillary blood samples from a subject between Day 0-Day 10 following a concussion. FIG. 17C shows a graph of NfL levels from capillary blood samples from a subject between Day 0-Day 10 following a concussion. FIG. 17D shows a graph of total-tau levels from capillary blood samples from a subject between Day 0-Day 10 following a concussion.

FIG. 18 shows a graph and significant correlation of capillary p-tau217 levels compared to venous p-tau217 levels using the Meso Scale Discovery (MSD) detection platform.

### DETAILED DESCRIPTION

Quantification of neuro biomarkers in venous blood reduces the need for cerebrospinal fluid (CSF) sampling for diagnostic confirmatory analyses. Currently, samples must be taken using venipuncture which typically requires a healthcare professional to perform sample collection. Once taken, samples typically need to be centrifuged within a few hours and plasma extracted and either shipped to the laboratory for analyses in dry ice, or stored, typically at ultra-low temperatures. The present disclosure provides an accurate and sensitive diagnosis method of neurodegenerative diseases or disorders from blood samples using a dried plasma spot card device (or dried blood drop card or dried blood spot card). Dry plasma spot (DPS) cards can separate soluble proteins from the cell fraction of whole blood, equivalent to the separation of standard bottled blood achieved using centrifuges, without the issues of protein concentration variation with hematocrit in dried blood spot cards (DBS). A small volume of blood, such as from a finger-prick sample may be applied to a DPS card, negating the need for formal venipuncture. This method and device provide an alternative means of blood collection that is less invasive and more convenient than venipuncture blood collection. Cards may then be stored or transported at room temperature prior to analysis. The ability to sample neurodegeneration-associated proteins in a quick, minimally invasive way, including in non-clinical settings (such as patients' homes) has significant implications for equity to diagnostics and great potential to improve personalized patient management.

The blood biomarkers such as axonal cytoskeletal protein neurofilament light (NfL) and astroglial marker glial fibrillary acidic protein (GFAP) are considered reliable measures of neuronal damage as a consequence of neurodegenerative (Zetterberg & Burnham, 2019), traumatic brain injury (TBI) (Graham et al., 2021; Shahim et al., 2020), cardiac arrest (PMID: 36877496) and neuroinflammatory conditions (Meier et al., 2023), with putative roles in screening, diagnosis, activity monitoring and assessment of treatment-response. In the context of Alzheimer's disease (AD), GFAP has been shown to increase concurrently with amyloid-PET signal in the brain (Benedet A L, et al., Translational Biomarkers in Aging and Dementia (TRIAD) study, Alzheimer's and Families (ALFA) study, and BioCogBank Paris Lariboisière cohort. Differences Between Plasma and Cerebrospinal Fluid Glial Fibrillary Acidic Protein Levels Across the Alzheimer Disease Continuum. JAMA Neurol. 2021 Dec. 1; 78(12):1471-1483) and increase >10 years prior to estimated disease onset in genetically determined AD (Montoliu-Gaya L, et al. Plasma and cerebrospinal fluid glial fibrillary acidic protein levels in adults with Down syndrome: a longitudinal cohort study. EBioMedicine. 2023 April; 90:104547; O'Connor A, et al. Plasma GFAP in presymptomatic and symptomatic familial Alzheimer's disease: a longitudinal cohort study. J Neurol Neurosurg Psychiatry. 2023 January; 94(1):90-92). In addition to these two biomarkers, testing additional neuro biomarkers such as phosphorylated proteins can also provide a more accurate and/or earlier diagnosis result of patients who experience cognitive decline, who have a neurodegenerative condition, or who are at elevated risk for developing a neurodegenerative condition.

Tau is a protein that is predominantly found in non-myelinated axon such as in brain neurons, and tau helps stabilize the internal skeleton of these neurons. Abnormal or excessive phosphorylation of tau has been associated with transformation of pathologically normal tau molecules into paired-helical-filament (PHF) tau and neurofibrillary tangles (NFTs) indicative of various tauopathy pathologies. Accumulation of NFTs together with beta-amyloid plaques are hallmarks the neurodegenerative condition Alzheimer's disease, and progressive accumulation of NFTs and beta-amyloid plaques is associated with progressive cognitive decline in AD patients. Protein phosphorylation is a posttranslational modification occurring by the addition of a phosphate group to specific amino acid residues on a protein. Hyperphosphorylation of tau can cause tau protein to detach from the microtubules, thereby destabilizing internal skeleton and compromising axonal transport. Tau can be phosphorylated at different amino acid residues, for examples, at threonine 217 (pTau-217) or at threonine 181 (pTau-181). Phosphorylated-tau proteins comprising phosphorylated Tau-181 (pTau-181), phosphorylated Tau-217 (pTau-217), phosphorylated Tau-231 (pTau-231), phosphorylated Tau-212 (pTau-212), or phosphorylated Tau-212+217 (pTau-217+) can be used as neuro biomarkers to diagnose cognitive decline or to diagnose a neurodegenerative condition comprising a tauopathy such as Alzheimer's disease (AD). Brain-derived tau (BD-Tau) can also be used as an additional informative neuro biomarker.

Accurate detection and quantification of phosphorylated proteins such as phosphorylated tau proteins from biological samples such as blood samples can be challenging. Phosphorylated tau proteins may be present in varying concentrations from biological samples derived from a subject depending on the source of the biological sample. For instance, phosphorylated tau proteins may be present at a higher concentration in a CSF sample from a subject, than within an blood sample collected by venipuncture blood sample or from a capillary blood sample derived from the subject. In another instance, phosphorylated tau proteins may be present at a higher concentration within a blood sample collected by venipuncture compared to a capillary blood sample. In terms of ease of sample collection, undergoing a venous blood sample collection or a capillary blood sample collection is easier and less disruptive on the subject than undergoing a CSF sample collection procedure. Collecting a capillary blood sample is further easier and less disruptive to the subject than undergoing a venipuncture sample collection procedure. A capillary blood sample may be collected using a finger-prick and collection of the biological sample comprising capillary blood on one or more DPS cards. The ease of collecting a capillary blood sample from a subject, including remote sample collection or self-collection by the subject using one or more DPS cards, necessitates that a capillary blood sample is analyzed with sufficient specificity and sensitivity for one or more selected neuro biomarkers to produce an informative diagnostic test result to achieve the utility of remote sample collection. Developing neuro biomarkers assays with sufficient specificity and sensitivity using a capillary blood sample is paramount to achieve the ease and utility of remote sample collection and informative diagnosis of a neurological or neurodegenerative condition.

Described herein, the present disclosure also provides methods and compositions of protein extraction reagent (or protein extraction buffer) that can be used in a method to detect the presence of phosphorylated proteins from blood samples.

Provided herein are methods for determining protein levels of one or more biomarkers in a biological sample, the methods comprising: a) obtaining the biological sample, wherein the biological sample comprises a capillary blood sample derived from a subject; b) extracting proteins from the biological sample using a protein extraction reagent to obtain a protein-enriched extracted sample; c) performing an assay on the protein-enriched extracted sample, wherein the assay comprises determining a detected protein level of at least one of biomarkers: i) phosphorylated Tau-181 (pTau-181), ii) phosphorylated Tau-217 (pTau-217), iii) glial fibrillary acidic protein (GFAP), iv) neurofilament light chain (NFL), v) phosphorylated Tau-231 (pTau-231), vi) phosphorylated Tau-212 (pTau-212), vii) phosphorylated Tau-212+217 (pTau-217+), viii) brain-derived tau (BD-Tau), ix) 42-amino acid β amyloid (Aβ42) peptide, or x) 40-amino acid 1 amyloid (Aβ40) peptide.

Also provided herein are methods for determining protein levels of one or more biomarkers in a biological sample, the methods comprising: a) obtaining the biological sample, wherein the biological sample is a capillary blood sample derived from a subject; b) extracting proteins from the biological sample using a protein extraction reagent to obtain a protein-enriched extracted sample; c) performing an assay on the protein-enriched extracted sample, wherein the assay comprises determining a detected protein level of at least one of biomarkers: i) phosphorylated Tau-181 (pTau-181), ii) phosphorylated Tau-217 (pTau-217), iii) glial fibrillary acidic protein (GFAP), iv) neurofilament light chain (NFL), v) phosphorylated Tau-231 (pTau-231), vi) phosphorylated Tau-212 (pTau-212), vii) phosphorylated Tau-212+217 (pTau-217+), viii) brain-derived tau (BD-Tau), ix) 42-amino acid β amyloid (Aβ42) peptide, or x) 40-amino acid 3 amyloid (Aβ40) peptide.

Detection and quantification of phosphorylated protein in the biological sample can be performed using an optimized protein extraction reagent described herein. In some instances, this protein extraction reagent described herein is referred to as “High-Performance Protein Extraction Buffer.” In some embodiments, the protein extraction reagent comprises a buffered solution comprising tris(hydroxymethyl)aminomethane (Tris), sodium chloride (NaCl), bovine serum albumin (BSA), and a polysorbate nonionic surfactant. In some instances, the protein extract reagent can be used in a method of detection and quantification of specific proteins. In some instances, the protein extract reagent can be used in a method of detection and quantification of certain isoforms of specific proteins (e.g., detecting and quantifying one or more phosphorylated isoforms of a protein). In some instances, the protein extract reagent can be used in a method of detection and quantification of specific isoforms of phosphorylated tau proteins. In some instances, the protein extraction reagent can be used in a method of detection and quantification of other post-translational modifications of the proteins or biomarkers. In some instances, the protein extract reagent can be used in a method of identifying if a subject is at risk for having or developing a neurodegenerative disease. In some instances, the protein extract reagent can be used in a method for treating a neurodegenerative disease in a subject in need thereof.

In some embodiments, the protein extraction reagent comprises a buffered solution comprising phosphate buffered saline (PBS) comprising phosphate, NaCl, and potassium chloride (KCl). In some embodiments, the buffered solution further comprises a polysorbate nonionic surfactant.

In some embodiments of the protein extraction reagent, the buffered solution further comprises EDTA. In some embodiments, the buffered solution further comprises BSA. In some embodiments, the buffered solution further comprises magnesium chloride (MgCl₂), dextrose, and urea. In some embodiments, the buffered solution further comprises a TRU Block™ immunoassay blocker.

In various embodiments, the protein extraction reagent further comprises a preservative. In some embodiments, the preservative comprises ProClin™ 300. In some embodiments, the preservative comprises sodium azide. In some embodiments, the preservative comprises thimerosal. In some embodiments, the preservative comprises gentamicin. In some embodiments, the preservative comprises 5-chloro-2-methyl-4-isothiazolin-3-one (CMIT) and 2-methyl-4-isothiazolin-3-one (MIT). In some embodiments, the preservative comprises 3% of 5-chloro-2-methyl-4-isothiazolin-3-one (CMIT) and 2-methyl-4-isothiazloin-3-one (MIT) in a salt-free glycol solution further comprising an alkyl carboxylate stabilizer. In some embodiments, the protein extraction reagent further comprises EDTA.

In some embodiments of the protein extraction reagent, the polysorbate nonionic surfactant comprises either polyethylene glycol sorbitan monolaurate (Tween 20) or polyethylene glycol sorbitan monooleate (Tween 80). In some embodiments, the polysorbate nonionic surfactant comprises Tween 20. In some embodiments, the polysorbate nonionic surfactant comprises Tween 80.

In some embodiments of the protein extraction reagent, the buffered solution has a pH between about 7.0-9.0. In some embodiments, the buffered solution has a pH between about 7.1-9.0, about 7.2-9.0, about 7.3-9.0, about 7.4-9.0, about 7.5-9.0, about 7.6-9.0, about 7.7-9.0, about 7.8-9.0, about 7.9-9.0, about 8-9.0, about 8.1-9.0, about 8.2-9.0, about 8.3-9.0, about 8.4-9.0, about 8.5-9.0, about 8.6-9.0, about 8.7-9.0, about 8.8-9.0, about 8.9-9.0, about 7.0-8.5, about 7.0-8.4, about 7.0-8.2, about 7.0-8.0, about 7.0-7.8, about 7.0-7.6, about 7.0-7.5, about 7.0-7.4, about 7.0-7.2, about 7.2-8.9, about 7.2-8.8, about 7.2-8.6, about 7.2-8.4, about 7.2-8.2, about 7.2-8.0, about 7.2-7.8, about 7.2-7.6, about 7.2-7.4, about 7.4-8.9, about 7.4-8.8, about 7.4-8.6, about 7.4-8.4, about 7.4-8.2, about 7.4-8.0, about 7.4-7.8, about 7.4-7.6, about 7.6-8.9, about 7.6-9.0, about 7.6-8.8, about 7.6-8.4, about 7.6-8.2, about 7.6-8.0, about 7.6-7.8, about 7.8-8.9, about 7.8-8.8, about 7.8-8.6, about 7.8-8.4, about 7.8-8.2, about 7.8-8.0, about 8.0-8.9, about 8.0-8.8, about 8.0-8.6, about 8.0-8.4, about 8.0-8.2, about 8.2-8.9, about 8.2-8.7, about 8.2-8.5, about 8.2-8.3, about 8.4-8.9, about 8.4-8.7, about 8.4-8.5, about 8.6-8.9, about 8.6-8.8, about 8.6-8.7, about 8.7-8.9, about 8.7 to 8.8, or about 8.8-8.9.

In some embodiments of the protein extraction reagent, the buffered solution has a pH of about 7.0, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9.0. In some embodiments, the buffered solution has a pH of about 7.4. In some embodiments, the buffered solution has a pH of about 9.0.

In some embodiments of the protein extraction reagent, the buffered solution has a pH of at least about 7.0, at least about 7.1, at least about 7.2, at least about 7.3, at least about 7.4, at least about 7.5, at least about 7.6, at least about 7.7, at least about 7.8, at least about 7.9, at least about 8, at least about 8.1, at least about 8.2, at least about 8.3, at least about 8.4, at least about 8.5, at least about 8.6, at least about 8.7, at least about 8.8, at least about 8.9, or at least about 9.0.

In some embodiments of the protein extraction reagent, the buffered solution has a pH of less than about 7.0, less than about 7.1, less than about 7.2, less than about 7.3, less than about 7.4, less than about 7.5, less than about 7.6, less than about 7.7, less than about 7.8, less than about 7.9, less than about 8, less than about 8.1, less than about 8.2, less than about 8.3, less than about 8.4, less than about 8.5, less than about 8.6, less than about 8.7, less than about 8.8, less than about 8.9, less than about 9.0, or less than about 9.1.

In some embodiments of the protein extraction reagent, the TRU Block™ immunoassay blocker of the buffered solution is at concentration of about 0.10%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2%. In some embodiments, the TRU Block™ immunoassay blocker of the buffered solution is at concentration of about 1%. In some embodiments, the TRU Block™ immunoassay blocker of the buffered solution is at concentration between about 0.1%-3%, about 0.2%-3%, about 0.3%-3%, about 0.4%-3%, about 0.5%-3%, about 0.6%-3%, about 0.7%-3%, about 0.8%-3%, about 0.9%-3%, about 1%-3%, about 1.1%-3%, about 1.2%-3%, about 1.3%-3%, about 1.4%-3%, about 1.5%-3%, about 1.6%-3%, about 1.7%-3%, about 1.8%-3%, about 1.9%-3%, about 2%-3%, about 0.1%-1%, about 0.1%-1.2%, about 0.1%-1.4, about 0.1%-1.6%, about 0.1%-1.8%, about 0.1%-2%, about 0.4%-1%, about 0.4%-1.2%, about 0.4%-1.4%, about 0.4%-1.6%, about 0.4%-1.8%, about 0.4%-2%, about 0.4%-2.5%, about 0.6%-1%, about 0.6%-1.5%, about 0.6%-2%, about 0.6%-2.5%, about 1%-1.5%, about 1%-2%, about 1%-2.5%, about 1.5%-2%, or about 1.5%-2.5%.

In some embodiments, the TRU Block™ immunoassay blocker of the buffered solution is at concentration of at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.1%, at least about 1.2%, at least about 1.3%, at least about 1.4%, at least about 1.5%, at least about 1.6%, at least about 1.7%, at least about 1.8%, at least about 1.9%, or at least about 2%.

In some embodiments, the TRU Block™ immunoassay blocker of the buffered solution is at concentration of less than about 0.1%, less than about 0.2%, less than about 0.3%, less than about 0.4%, less than about 0.5%, less than about 0.6%, less than about 0.7%, less than about 0.8%, less than about 0.9%, less than about 1%, less than about 1.1%, less than about 1.2%, less than about 1.3%, less than about 1.4%, less than about 1.5%, less than about 1.6%, less than about 1.7%, less than about 1.8%, less than about 1.9%, or less than about 2%. In some embodiments the TRU Block™ immunoassay blocker of the buffered solution is at concentration of between about 1-100 μg/mL. In some embodiments the TRU Block™ immunoassay blocker of the buffered solution is at concentration of at least about 1 μg/mL, 2 μg/mL, 3 μg/mL, 4 μg/mL, 5 μg/mL, 6 μg/mL, 7 μg/mL, 8 μg/mL, 9 μg/mL, 10 μg/mL, 20 μg/mL, or 30 μg/mL. In some embodiments the TRU Block™ immunoassay blocker of the buffered solution is at concentration of less than about 50 μg/mL, 40 μg/mL, 30 μg/mL, 25 μg/mL, 20 μg/mL, 15 μg/mL, 12 μg/mL, 10 μg/mL, 9 μg/mL, 8 μg/mL, 6 μg/mL, or 5 μg/mL. In some embodiments the TRU Block™ immunoassay blocker of the buffered solution is at concentration of about 5 μg/mL. In some embodiments the TRU Block™ immunoassay blocker of the buffered solution is at concentration of about 7.5 μg/mL. In some embodiments the TRU Block™ immunoassay blocker of the buffered solution is at concentration of about 10 μg/mL. In some embodiments the TRU Block™ immunoassay blocker of the buffered solution is at concentration of about 15 μg/mL. In some embodiments the TRU Block™ immunoassay blocker of the buffered solution is at concentration of about 20 μg/mL.

Described herein are protein extraction reagents for use methods of ultra-sensitive quantification of levels of selected protein analytes that serve as biomarkers for neurological or neurodegenerative conditions. In some embodiments, the protein extraction reagent comprises a buffered solution. In some embodiments, the protein extraction reagent comprises between about 1 mM-110 mM Tris base, between about 50 mM-250 mM NaCl, between about 0.1%-3% BSA, between about 0.01%-2% of a polysorbate-type nonionic surfactant. In some embodiments, addition of ProClin™ 300 between about 0.01%-2% is optional. In some embodiments, addition of EDTA between about 1 mM-150 mM is optional. In some embodiments, addition of about 10 μg/mL TRU Block™ immunoassay blocker is optional.

In various embodiments, concentration of each component of the protein extraction reagent can be optimized. In some embodiments, the concentration of Tris is between about 1 mM to 110 mM, about 10 mM to 110 mM, about 20 mM to 110 mM, about 30 mM to 110 mM, about 40 mM to 110 mM, about 50 mM to 110 mM, about 60 mM to 110 mM, about 70 mM to 110 mM, about 80 mM to 110 mM, about 90 mM to 110 mM, or about 100 mM to 110 mM. In some embodiments, the concentration of Tris is about 1 mM, about 10 mM, about 20 mM, about 30 mM, about 40 mM, about 50 mM, about 60 mM, about 70 mM, about 80 mM, about 90 mM, or about 100 mM. In some embodiments, the concentration of Tris is at least about 1 mM, at least about 10 mM, at least about 20 mM, at least about 30 mM, at least about 40 mM, at least about 50 mM, at least about 60 mM, at least about 70 mM, at least about 80 mM, at least about 90 mM, or at least about 100 mM. In some embodiments, the concentration of Tris is less than about 10 mM, less than about 20 mM, less than about 30 mM, less than about 40 mM, less than about 50 mM, less than about 60 mM, less than about 70 mM, less than about 80 mM, less than about 90 mM, or less than about 100 mM. In some embodiments, the concentration of Tris is at least about 20 mM. In some embodiments, the concentration of Tris is less than about 15 mM. In some embodiments, the concentration of Tris is about 10 mM. In some embodiments, Trizma® base (121.14 molecular weight g/mol) is used as the form of Tris base.

In various embodiments, the concentration of NaCl is between about 50 mM to about 250 mM, about 60 mM to about 250 mM, about 70 mM to about 250 mM, about 80 mM to about 250 mM, about 90 mM to about 250 mM, about 100 mM to about 250 mM, about 110 mM to about 250 mM, about 120 mM to about 250 mM, about 130 mM to about 250 mM, about 140 mM to about 250 mM, about 150 mM to about 250 mM, about 160 mM to about 250 mM, about 170 mM to about 250 mM, about 180 mM to about 250 mM, about 190 mM to about 250 mM, or about 200 mM to about 250 mM. In some embodiments, the concentration of NaCl is about 50 mM, about 60 mM, about 70 mM, about 80 mM, about 90 mM, about 100 mM, about 110 mM, about 120 mM, about 130 mM, about 137 mM, about 140 mM, about 150 mM, about 160 mM, about 170 mM, about 180 mM, about 190 mM, or about 200 mM. In some embodiments, the concentration of NaCl is at least about 50 mM, at least about 60 mM, at least about 70 mM, at least about 80 mM, at least about 90 mM, at least about 100 mM, at least about 110 mM, at least about 120 mM, at least about 130 mM, at least about 140 mM, at least about 150 mM, at least about 160 mM, at least about 170 mM, at least about 180 mM, at least about 190 mM, or at least about 200 mM. In some embodiments, the concentration of NaCl is less than about 50 mM, less than about 60 mM, less than about 70 mM, less than about 80 mM, less than about 90 mM, less than about 100 mM, less than about 110 mM, less than about 120 mM, less than about 130 mM, less than about 140 mM, less than about 150 mM, less than about 160 mM, less than about 170 mM, less than about 180 mM, less than about 190 mM, or less than about 200 mM.

In various embodiments, the concentration of BSA is between about 0.1%-3%, about 0.2%-3%, about 0.3%-3%, about 0.4%-3%, about 0.5%-3%, about 0.6%-3%, about 0.7%-3%, about 0.8%-3%, about 0.9%-3%, about 1%-3%, about 1.1%-3%, about 1.2% 3%, about 1.3%-3%, about 1.4%-3%, about 1.5%-3%, about 1.6%-3%, about 1.7%-3%, about 1.8%-3%, about 1.9%-3%, or about 2%-3%. In some embodiments, the concentration of BSA is about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2%. In some embodiments, the concentration of BSA is at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.1%, at least about 1.2%, at least about 1.3%, at least about 1.4%, at least about 1.5%, at least about 1.6%, at least about 1.7%, at least about 1.8%, at least about 1.9%, or at least about 2%. In some embodiments, the concentration of BSA is less than about 0.1%, less than about 0.2%, less than about 0.3%, less than about 0.4%, less than about 0.5%, less than about 0.6%, less than about 0.7%, less than about 0.8%, less than about 0.9%, less than about 1%, less than about 1.1%, less than about 1.2%, less than about 1.3%, less than about 1.4%, less than about 1.5%, less than about 1.6%, less than about 1.7%, less than about 1.8%, less than about 1.9%, or less than about 2%.

In various embodiments, the concentration of ProClin™ 300 is about 0.010, about 0.05%, about 0.1%, about 0.2%, about 0.3%, about 0.4%, about 0.5%, about 0.6%, about 0.7%, about 0.8%, about 0.9%, about 1%, about 1.2%, about 1.4%, about 1.5%, about 1.6%, about 1.8%, or about 2%. In some embodiments, the concentration of ProClin™ 300 is between about 0.01%-about 2%, about 0.05%-about 2%, about 0.1%-about 2%, about 0.2%-about 2%, about 0.3%-about 2%, about 0.4%-about 2%, about 0.5%-about 2%, about 0.6%-about 2%, about 0.7%-about 2%, about 0.8%-about 2%, about 0.9%-about 2%, or about 1%-about 2%. In some embodiments, the concentration of ProClin™ 300 is at least about 0.01%, at least about 0.05%, at least about 0.1%, at least about 0.2%, at least about 0.3%, at least about 0.4%, at least about 0.5%, at least about 0.6%, at least about 0.7%, at least about 0.8%, at least about 0.9%, at least about 1%, or at least about 2%. In some embodiments, the concentration of ProClin™ 300 is less than about 0.01%, less than about 0.05%, less than about 0.1%, less than about 0.2%, less than about 0.3%, less than about 0.4%, less than about 0.5%, less than about 0.6%, less than about 0.7%, less than about 0.8%, less than about 0.9%, less than about 1%, or less than about 2%.

In various embodiments, the concentration of Tween 20 is about 0.01%, about 0.05%, about 0.1%, about 0.2%, about 0.3%, about 0.4%, about 0.5%, about 0.6%, about 0.7%, about 0.8%, about 0.9%, about 1%, or about 2%. In some embodiments, the concentration of Tween20 is between about 0.01%-about 2%, about 0.05%-about 2%, about 0.1%-about 2%, about 0.2%-about 2%, about 0.3%-about 2%, about 0.4%-about 2%, about 0.5%-about 2%, about 0.6%-about 2%, about 0.7%-about 2%, about 0.8%-about 2%, about 0.9%-about 2%, or about 1%-about 2%. In some embodiments, the concentration of Tween20 is at least about 0.01%, at least about 0.05%, at least about 0.1%, at least about 0.2%, at least about 0.3%, at least about 0.4%, at least about 0.5%, at least about 0.6%, at least about 0.7%, at least about 0.8%, at least about 0.9%, at least about 1%, or at least about 2%. In some embodiments, the concentration of Tween20 is less than about 0.01%, less than about 0.05%, less than about 0.1%, less than about 0.2%, less than about 0.3%, less than about 0.4%, less than about 0.5%, less than about 0.6%, less than about 0.7%, less than about 0.8%, less than about 0.9%, less than about 1%, or less than about 2%.

In various embodiments, the concentration of EDTA is between about 1 mM-150 mM, about 5 mM-150 mM, about 10 mM-150 mM, about 20 mM-150 mM, about 30 mM-150 mM, about 40 mM-150 mM, about 50 mM-150 mM, about 60 mM-150 mM, about 70 mM-150 mM, about 80 mM-150 mM, about 90 mM-150 mM, or about 100 mM-150 mM.

In some embodiments, the concentration of EDTA is 1 mM, about 5 mM, about 10 mM, about 20 mM, about 30 mM, about 40 mM, about 50 mM, about 60 mM, about 70 mM, about 80 mM, about 90 mM, or about 100 mM. In some embodiments, the concentration of EDTA is at least about 1 mM, at least about 5 mM, at least about 10 mM, at least about 20 mM, at least about 30 mM, at least about 40 mM, at least about 50 mM, at least about 60 mM, at least about 70 mM, at least about 80 mM, at least about 90 mM, or at least about 100 mM. In some embodiments, the concentration of EDTA is less than about 1 mM, less than about 5 mM, less than about 10 mM, less than about 20 mM, less than about 30 mM, less than about 40 mM, less than about 50 mM, less than about 60 mM, less than about 70 mM, less than about 80 mM, less than about 90 mM, or less than about 100 mM.

In various embodiments, the protein extraction reagent comprises about a phosphate buffered solution of about 10 mM Na₂HPO₄, about 1.8 mM KH₂PO₄, about 137 mM NaCl, about 2.7 mM KCl, about 0.5% BSA, about 0.05% ProClin™ 300, and about 0.1% Tween 20. In various embodiments, the protein extraction reagent comprises about a phosphate buffered solution of about 10 mM Na₂HPO₄, about 1.8 mM KH₂PO₄, about 137 mM NaCl, about 2.7 mM KCl, about 0.5% BSA, about 0.05% ProClin™ 300, about 10 μg/mL TRU Block™, about 0.1% Tween 20, and about 5 mM EDTA.

In various embodiments, the protein extraction reagent comprises about a borate buffered solution of about 0.05M sodium tetra borate mixed with 0.1M HCl a pH range of between about 7.8 to about 9.0, about 0.5% BSA, about 0.05% ProClin™ 300, and about 0.1% Tween 20. In various embodiments, the protein extraction reagent comprises about a borate buffered solution of about 0.05M sodium tetra borate mixed with 0.1M HCl a pH range of between about 7.8 to about 9.0, about 0.5% BSA, about 0.05% ProClin™ 300, and about 0.1% Tween 20, and about 5 mM EDTA.

In various embodiments, the protein extraction reagent comprises about 10 mM Tris, about 125 mM KCl, about 0.5% BSA, about 0.05% ProClin™ 300, and about 0.1% Tween 20. In various embodiments, the protein extraction reagent comprises about 10 mM Tris, about 125 mM KCl, about 0.5% BSA, about 10 μg/mL TRU Block™, about 0.1% Tween 20, and about 5 mM EDTA.

In various embodiments, the protein extraction reagent comprises about 20 mM Tris, about 137 mM NaCl, about 1% BSA, about 0.05% ProClin™ 300, and about 0.11% Tween 20. In some embodiments, addition of 10 μg/mL TRU Block™ is optional. In various embodiments, the protein extraction reagent comprises about 20 mM Tris, about 137 mM NaCl, about 1% BSA, about 0.05% ProClin™ 300, about 0.1% Tween 20, and about 5 mM EDTA. In some embodiments, the protein extraction reagent has a pH of about 9.0.

In various embodiments, the protein extraction reagent comprises about 0.01M Tris, about 0.15M NaCl, about 2% BSA, and about 0.10% Tween-20. In various embodiments, the protein extraction reagent comprises about 0.01M Tris, about 0.15M NaCl, about 2% BSA, about 0.10% Tween-20, and about 5 mM EDTA. In some embodiments, the protein extraction reagent further comprises ProClin™ 300. In some embodiments, the ProClin™ 300 is present at about 0.05%. In some embodiments, the protein extraction reagent further comprises TRU Block™ immunoassay blocker. In some embodiments, TRU Block™ immunoassay blocker is present at about 10 μg/mL.

In various embodiments, the protein extraction reagent comprises at least about 5 mM Tris base, at least about 100 mM NaCl, at least about 1% BSA, and a least about 0.05% of a polysorbate nonionic surfactant. In some embodiments, the polysorbate nonionic surfactant comprise Tween-20. In some embodiments, the polysorbate nonionic surfactant comprise Tween-80. In some embodiments, the protein extraction reagent further comprises at least about 2.5 mM EDTA. In some embodiments, the protein extraction reagent has a pH of at least about 8.0. In some embodiments, the protein extraction reagent further comprises ProClin™ 300. In some embodiments, the ProClin™ 300 is present at about 0.05%.

In various embodiments, the protein extraction reagent comprises less than about 15 mM Tris base, than less about 200 mM NaCl, less than about 2.5% BSA, and less than about 0.15% of a polysorbate nonionic surfactant. In some embodiments, the polysorbate nonionic surfactant comprise Tween-20. In some embodiments, the polysorbate nonionic surfactant comprise Tween-80. In some embodiments, the protein extraction reagent further comprises less than about 10 mM EDTA. In some embodiments, the protein extraction reagent has a pH of about 9.0. In some embodiments, the protein extraction reagent further comprises ProClin™ 300. In some embodiments, the ProClin™ 300 is present at about 0.05%. In some embodiments, the protein extraction reagent consists essentially of 10 mM Tris base, 150 mM NaCl, 2% BSA, 0.10% Tween-20, and 5 mM EDTA. In some embodiments, the protein extraction reagent consists essentially of 10 mM Tris base, 150 mM NaCl, 2% BSA, 0.10% Tween-20, 5 mM EDTA, and 0.05% ProClin™ 300. In some embodiments, addition of TRU Block™ immunoassay blocker is optional. In some embodiments, TRU Block™ immunoassay blocker is present at about 10 μg/mL.

In various embodiments, the protein extraction reagent comprises less than about 25 mM Tris base, than less about 175 mM NaCl, less than about 1.5% BSA, and less than about 0.15% of a polysorbate nonionic surfactant. In some embodiments, the polysorbate nonionic surfactant comprise Tween-20. In some embodiments, the polysorbate nonionic surfactant comprise Tween-80. In some embodiments, the protein extraction reagent further comprises less than about 10 mM EDTA. In some embodiments, the protein extraction reagent has a pH of about 9.0. In some embodiments, the protein extraction reagent further comprises ProClin™ 300. In some embodiments, the ProClin™ 300 is present at about 0.05%. In some embodiments, the protein extraction reagent consists essentially of 20 mM Tris, 137 mM NaCl, 1% BSA, 0.05% ProClin™ 300, 0.1% Tween-20, 5 mM EDTA, and has a pH of about 9.0. In some embodiments, addition of TRU Block™ immunoassay blocker is optional. In some embodiments, TRU Block™ immunoassay blocker is present at about 10 μg/mL.

In various embodiments, the protein extraction reagent comprises at least about 15 mM Tris base, at least about 125 mM NaCl, at least about 0.75% BSA, and at least about 0.05% of a polysorbate nonionic surfactant. In some embodiments, the polysorbate nonionic surfactant comprise Tween-20. In some embodiments, the polysorbate nonionic surfactant comprise Tween-80. In some embodiments, the protein extraction reagent further comprises less than about 10 mM EDTA. In some embodiments, the protein extraction reagent has a pH of about 9.0. In some embodiments, the protein extraction reagent further comprises ProClin™ 300. In some embodiments, the ProClin™ 300 is present at about 0.05%. In some embodiments, addition of TRU Block™ immunoassay blocker is optional. In some embodiments, TRU Block™ immunoassay blocker is present at about 10 μg/mL.

In various embodiments, the step b) further comprises incubating the biological samples for 30 minutes. In some embodiments, the step b) further comprises incubating the biological samples for about 10 minutes, about 20 minutes, about 30 minutes, about 40 minutes, about 50 minutes, about 60 minutes, about 70 minutes, about 80 minutes, about 90 minutes, about 100 minutes, about 110 minutes, or about 120 minutes. In some embodiments, the step b) further comprises incubating the biological samples for at least 10 minutes, at least 20 minutes, at least 30 minutes, at least 40 minutes, at least 50 minutes, at least 60 minutes, at least 70 minutes, at least 80 minutes, at least 90 minutes, at least 100 minutes, at least 110 minutes, or at least 120 minutes. In some embodiments, the step b) further comprises incubating the biological samples for at least 30 minutes. In some embodiments, the step b) further comprises incubating the biological samples for less than 10 minutes, less than 20 minutes, less than 30 minutes, less than 40 minutes, less than 50 minutes, less than 60 minutes, less than 70 minutes, less than 80 minutes, less than 90 minutes, less than 100 minutes, less than 110 minutes, or less than 120 minutes.

In various embodiments, the step b) further comprises transferring the biological sample to a quantification plate. The quantification plate is used for sample analysis. The biological sample can be centrifuged through the filter plate and collected in a quantification plate for analysis. In some embodiments, the biological sample is transferred to the quantification plate via centrifugation through a filter plate. In some embodiments, the quantification plate is compatible for Simoa® analysis (Quanterix). In some embodiments, the Simoa® analysis is capable of analyzing and quantitating p-tau181, p-tau212, p-tau217, p-tau217+, or p-tau231, or any combination thereof. In some embodiments, the Simoa® analysis utilizes an anti-tau antibody from ALZpath, Janssen, Quanterix, ADx, or UGOT, or any combination thereof. In some embodiments, the quantification plate is compatible for Meso Scale Discovery (MDS) analysis. In some embodiments, the quantification plate is compatible for Lumipulse® immunoassay analysis (Fujirebio). In some embodiments, the quantification plate is compatible for Elecsys Cobas® e801 immunohistochemistry analysis (Roche Diagnostics). In some embodiments, the quantification plate is compatible for Ella Automated Immunoassay System analysis (Bio-Techne). In some embodiments, the quantification plate is compatible for ARCHITECT Immunoassay System analysis (Fisher Scientific). In some embodiments, the quantification plate is compatible for Atellica® Solution Immunoassay analysis (Siemens Heathineers). In some embodiments, the quantification plate is compatible for ARGO™ HT System Immunoassay analysis (Alamar Biosciences). In some embodiments, the ARGO™ HT System is an ARGO™ HT System p-tau181 analysis system. In some embodiments, the ARGO™ HT System is an ARGO™ HT System p-tau217 analysis system. In some embodiments, the ARGO™ HT System is an ARGO™ HT System p-tau231 analysis system. In some embodiments, the ARGO™ HT System is an ARGO™ HT System p-tau212 analysis system. In some embodiments, the ARGO™ HT System is capable of analyzing and quantitating p-tau181, p-tau212, p-tau217, or p-tau231, or any combination thereof. In some embodiments, the quantification plate is compatible for the Olink® immunoassay platform proximity extension assay system (Thermo Fisher Scientific). In some embodiments, the quantification plate is compatible for mass spectrometry analysis. In some embodiments, the quantitation plate is set up for singleplex analysis using one specific detection reagent. In some embodiments, the quantitation plate is set up for 2-plex analysis using two specific detection reagents. In some embodiments, the 2-plex analysis system is a Quanterix system (NfL and GFAP). In some embodiments, the quantitation plate is set up for 3-plex analysis using three specific detection reagents. In some embodiments, the quantitation plate is set up for 4-plex analysis using four specific detection reagents. In some embodiments, the 4-plex analysis system is a Quanterix system (NfL, GFAP, t-tau & UCHL1). In some embodiments, the quantitation plate is set up for multiplexed analysis using two, three, four, five, six, seven, eight, nine, or ten, or more specific detection reagents.

In various embodiments, the assay in step c) comprises an immunoassay. In some embodiments, the immunoassay comprises Western blot analysis, dot blot analysis, flow cytometry-based immunoassay, enzyme-linked immunosorbent assay (ELISA), lateral flow immunoassay, radioimmunoassay (RIA), competition immunoassay, dual antibody sandwich assay, chemiluminescent assay, bioluminescent assay, fluorescent assay, or agglutination assay. In some embodiments, the immunoassay comprises: i) single-molecule immunosorbent assay (Simoa®), ii) Meso Scale Discovery high performance electrochemiluminescence (MSD) assay, iii) Lumipulse® assay, iv) Elecsys® assay, v) Ella® Immunoassay (ProteinSimple, Bio-Techne), vi) ARCHITECT® Immunoassay (Abbott), vii) Atellica® Immunoassay (Siemens Healthineers), ARGO™ Immunoassay (Alamar), or ix) Olink® immunoassay platform proximity extension assay system (Thermo Fisher Scientific. In some embodiments, the immunoassay comprises Simoa®. In some embodiments, the assay comprises mass spectrometry. In some embodiments, the assay comprises use of the Olink® immunoassay platform proximity extension assay system (Thermo Fisher Scientific).

Various types of blood sample obtained from different sources can be used in the methods described herein. In some embodiments, the blood sample is obtained from a human. In some embodiments, the human is referred to as a subject or a patient. In some embodiments, the subject is at risk for developing a neurodegenerative condition. In some embodiments, the subject is at risk for developing a tauopathy. In some embodiments, the subject is at risk for developing Alzheimer's disease (AD). In some embodiments, the subject is at risk for developing clinical features of a neurodegenerative condition comprising cognitive decline. In some embodiments, the subject has not undergone a clinical evaluation for presence of a neurodegenerative condition. In some embodiments, the subject has undergone a clinical evaluation for presence of a neurodegenerative condition. In some embodiments, the subject has not been diagnosed with a neurodegenerative condition. In some embodiments, the subject has been diagnosed with a neurodegenerative condition. In some embodiments, the subject has not been determined to have pre-clinical AD. In some embodiments, the subject has been determined to have pre-clinical AD. In some embodiments, the subject has not been determined to have prodromal AD. In some embodiments, the subject has been determined to have prodromal AD. In some embodiments, the subject has not been determined to have AD dementia. In some embodiments, the subject has been determined to have AD dementia. In some embodiments, the subject has not been determined to be amyloid-β positive. In some embodiments, the subject has been determined to be amyloid-β positive. In some embodiments, the subject has not been determined to exhibit mild cognitive impairment. In some embodiments, the subject has been determined to exhibit mild cognitive impairment. In some embodiments, the subject has not been determined to exhibit moderate cognitive impairment. In some embodiments, the subject has been determined to exhibit moderate cognitive impairment. In some embodiments, the subject has not been determined to exhibit moderate-to-severe cognitive impairment. In some embodiments, the subject has been determined to exhibit moderate-to-severe cognitive impairment. In some embodiments, the subject has not been determined to exhibit dementia. In some embodiments, the subject has been determined to exhibit dementia. In some embodiments, the subject is a rodent. In some embodiments, the subject is a rat or a mouse. In some embodiments, the mouse comprises a genetic background comprising a transgene-induced tauopathy.

Various types of blood sample obtained from different sources can be used in the methods described herein. In some embodiments, the blood sample comprises a capillary blood sample or a venous blood sample. In some embodiments, the blood sample comprises a capillary blood sample. In some embodiments, the blood sample comprises a venous blood sample. In some embodiments, the blood sample is a capillary blood sample. In some embodiments, the blood sample is a venous blood sample. In some embodiments, the blood sample can be obtained using lancet or via venipuncture. In some embodiments, the capillary blood sample comprises a plurality of drops of blood. In some embodiments, the capillary blood sample comprises a single drop of blood. In some embodiments, the capillary blood sample comprises about 1 drop of blood, about 2 drops of blood, about 3 drops of blood, about 4 drops of blood, or about 5 drops of blood. In some embodiments, the capillary blood sample comprises about 1 drop of blood to about 10 drops of blood, about 2 drops of blood to about 10 drops of blood, about 3 drops of blood to about 10 drops of blood, about 4 drops of blood to about 10 drops of blood, or about 5 drops of blood to about 10 drops of blood. In some embodiments, the capillary blood sample comprises at least about 1 drop of blood, at least about 2 drops of blood, at least about 3 drops of blood, at least about 4 drops of blood, or at least about 5 drops of blood. In some embodiments, the capillary blood sample comprises less than about 1 drop of blood, less than about 2 drops of blood, less than about 3 drops of blood, less than about 4 drops of blood, or less than about 5 drops of blood.

In some embodiments, the capillary blood sample has been deposited on a blood sample collection card to create a spotted blood sample. As described herein, the blood sample collection card is referred to as “dried blood drop card” or “dried plasma card” or “dry plasma spot card”. A dry plasma spot card is referred to as a DPS card. In some embodiments, the DPS card efficiency separates plasma from the biological sample and stores the plasma portion for analysis at a later time.

In various embodiments, the volume of the spotted blood sample is less than about 100 μL. In some embodiments, the volume of the spotted blood sample is about 10 μL, about 20 μL, about 30 μL, about 40 μL, about 50 μL, about 60 μL, about 70 μL, about 80 μL, about 90 μL, about 100 μL, about 110 μL, about 120 μL, about 130 μL, about 140 μL, about 150 μL, about 160 μL, about 170 μL, about 180 μL, about 190 μL, or about 200 μL. In some embodiments, the volume of the spotted blood sample is about 10 μL to about 250 μL, about 20 μL to about 250 μL, about 30 μL to about 250 μL, about 40 μL to about 250 μL, about 50 μL to about 250 μL, about 60 μL to about 250 μL, about 70 μL to about 250 μL, about 80 μL to about 250 μL, about 90 μL to about 250 μL, about 100 μL to about 250 μL, about 110 μL to about 250 μL, about 120 μL to about 250 μL, about 130 μL to about 250 μL, about 140 μL to about 250 μL, about 150 μL to about 250 μL, about 160 μL to about 250 μL, about 170 μL to about 250 μL, about 180 μL to about 250 μL, about 190 μL to about 250 μL, or about 200 μL to about 250 μL. In some embodiments, the volume of the spotted blood sample is at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 110 μL, at least about 120 μL, at least about 130 μL, at least about 140 μL, at least about 150 μL, at least about 160 μL, at least about 170 μL, at least about 180 μL, at least about 190 μL, or at least about 200 μL. In some embodiments, the volume of the spotted blood sample is less than about 10 μL, less than about 20 μL, less than about 30 μL, less than about 40 μL, less than about 50 μL, less than about 60 μL, less than about 70 μL, less than about 80 μL, less than about 90 μL, less than about 100 μL, less than about 110 μL, less than about 120 μL, less than about 130 μL, less than about 140 μL, less than about 150 μL, less than about 160 μL, less than about 170 μL, less than about 180 μL, less than about 190 μL, or less than about 200 μL.

In various embodiments, the blood sample collection card comprises a plasma separation card. In some embodiments, the plasma separation card generates cell-free plasma from the spotted blood sample to use as the biological sample in step a). In some embodiments, the cell-free plasma is collected on at least two plasma separation membranes.

In some embodiments, the at least two plasma separation membranes are housed within a DPS card. In some embodiments, the DPS card protects the at least two plasma separation membranes to allow a remotely collected blood sample to be shipped to laboratory location for protein biomarker quantification and analysis.

In some embodiments, at least one of the at least two plasma separation membrane undergoes protein extraction in step b) using a protein extraction reagent comprising a buffered solution comprising Tris, NaCl, BSA, and a polysorbate nonionic surfactant; wherein at least one of biomarkers: i) pTau-181, ii) pTau-217, v) pTau-231, vi) pTau-212, vii) pTau-217+, or viii) BD-Tau is assayed to determine the detected protein level. In some embodiments, at least one of the at least two plasma separation membrane undergoes protein extraction in step b) using a protein extraction reagent comprising a buffered solution comprising Tris, NaCl, BSA, and a polysorbate nonionic surfactant; wherein at least one of biomarkers: i) pTau-181, ii) pTau-217, or v) pTau-231 is assayed to determine the detected protein level. In some embodiments, the determined detected protein level is a quantitative measure of protein level for one or more biomarkers in the sample. In some embodiments, at least one of the at least two plasma separation membrane undergoes protein extraction in step b) using “High-Performance Protein Extraction Buffer”. In various embodiments, the protein extraction reagent further comprises EDTA. In various embodiments, the protein extraction reagent further comprises ProClin™ 300. In various embodiments, the protein extraction reagent further comprises TRU Block™ immunoassay blocker. In some embodiments, the TRU Block™ immunoassay blocker is present in a concentration of about 1%. In some embodiments, the TRU Block™ immunoassay blocker is present at about 10 μg/mL. In some embodiments, ii) pTau-217 is assayed to determine the detected protein level. In some embodiments, at least two of i) pTau-181, ii) pTau-217, v) pTau-231, vi) pTau-212, vii) pTau-217+, or viii) BD-Tau are assayed to determine the detected protein level. In some embodiments, the determined detected protein level is a quantitative measure of protein level for one or more biomarkers in the sample.

In various embodiments, at least one of the at least two plasma separation membrane undergoes protein extraction in step b) using a protein extraction reagent comprising a buffered solution comprising PBS; wherein at least one of biomarkers: iii) GFAP, iv) NFL, vi) Aβ42 peptide, or vii) Aβ40 peptide is assayed to determine the detected protein level. In some embodiments, the detected protein level of the biomarkers: ii) phosphorylated Tau-217 (pTau-217), iii) glial fibrillary acidic protein (GFAP), and iv) neurofilament light chain (NFL), are determined in step c). In some embodiments, the detected protein level of the biomarkers: i) phosphorylated Tau-181 (pTau-181), ii) phosphorylated Tau-217 (pTau-217), iii) glial fibrillary acidic protein (GFAP), and iv) neurofilament light chain (NFL), are determined in step c). In some embodiments, the determined detected protein level is a quantitative measure of protein level for one or more biomarkers in the sample. In various embodiments, the capillary blood sample is dried prior to step b). In some embodiments, the capillary blood sample is dried prior to step b) for at least about 2, 3, 4, 5, 6, 7, 8, 9, 10, 11, 12, 13, 14, 15, 16, 17, 18, 19, or 20, or more than 20 minutes.

In various embodiments, the biological sample is stored for up to about two weeks at ambient temperature prior to extracting proteins in step b). In some embodiments, the biological sample is stored for about 1 days, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days at ambient temperature prior to extracting proteins in step b). In some embodiments, the biological sample is stored for at least about 1 days, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or at least about 31 days. In some embodiments, the biological sample is stored for less than 1 days, less than about 2 days, less than about 3 days, less than about 4 days, less than about 5 days, less than about 6 days, less than about 7 days, less than about 8 days, less than about 9 days, less than about 10 days, less than about 11 days, less than about 12 days, less than about 13 days, less than about 14 days, less than about 15 days, less than about 16 days, less than about 17 days, less than about 18 days, less than about 19 days, less than about 20 days, less than about 21 days, less than about 22 days, less than about 23 days, less than about 24 days, less than about 25 days, less than about 26 days, less than about 27 days, less than about 28 days, less than about 29 days, less than about 30 days, or less than about 31 days. In some embodiments, the biological sample is stored for between 1 day to 31 days, 2 days to 31 days, 3 days to 31 days, 4 days to 31 days, 5 days to 31 days, 6 days to 31 days, 7 days to 31 days, 8 days to 31 days, 9 days to 31 days, 10 days to 31 days, 11 days to 31 days, 12 days to 31 days, 13 days to 31 days, 14 days to 31 days, 15 days to 31 days, 16 days to 31 days, 17 days to 31 days, 18 days to 31 days, 19 days to 31 days, 20 days to 31 days, 21 days to 31 days, 22 days to 31 days, 23 days to 31 days, 24 days to 31 days, 25 days to 31 days, 26 days to 31 days, 27 days to 31 days, 28 days to 31 days, 29 days to 31 days, or 30 days to 31 days, at ambient temperature prior to extracting proteins in step b).

In some embodiments, the biological sample is stored for up to about two months at about 4° C. prior to extracting proteins in step b). In some embodiments, the biological sample is stored for up to about 1 month, about 2 months, about 3 months, about 4 months, about 5 months, about 6 months, about 7 months, about 8 months, about 9 months, about 10 months, about 11 months, or about 12 months, at about 4° C. prior to extracting proteins in step b). In some embodiments, the biological sample is stored for up to at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months, at least about 4° C. prior to extracting proteins in step b). In some embodiments, the biological sample is stored for less than about 1 month, less than about 2 months, less than about 3 months, less than about 4 months, less than about 5 months, less than about 6 months, less than about 7 months, less than about 8 months, less than about 9 months, less than about 10 months, less than about 11 months, or less than about 12 months, at less than about 4° C. prior to extracting proteins in step b). In some embodiments, the biological sample is stored for between about 1 month to about 13 months, about 2 months to about 13 months, about 3 months to about 13 months, about 4 months to about 13 months, about 5 months to about 13 months, about 6 months to about 13 months, about 7 months to about 13 months, about 8 months to about 13 months, about 9 months to about 13 months, about 10 months to about 13 months, about 11 months to about 13 months, or about 12 months to about 13 months, at about 4° C. prior to extracting proteins in step b).

In some embodiments, the detected protein level of each biomarker tested derived from the capillary blood sample is significantly correlated with a determined protein level of each respective biomarker tested in plasma isolated from a venous blood sample from the subject. In some embodiments, two or more biological samples collected from the same subject were compared between sources of the two or more biological samples. In some embodiments, the detected protein level of each biomarker tested derived from the capillary blood sample is significantly correlated with a determined protein level of each respective biomarker tested in a cerebrospinal fluid (CSF) sample from the subject. In some embodiments, the detected protein level of each biomarker tested derived from the capillary blood sample were quantified using a single molecule array (Simoa®) assay. In some embodiments, the capillary blood sample was collected using one or more DPS cards. In some embodiments, the detected protein level of each biomarker tested derived from the capillary blood sample collected using one or more DPS cards quantified using Simoa® assay were correlated to results from assaying plasma derived from a venous blood sample from the subject. In some embodiments, strong correlations between biomarker concentrations in plasma and finger-prick DPS samples are determined to be present. In some embodiments, the detected protein level of each biomarker tested derived from the capillary blood sample collected using one or more DPS cards quantified using Simoa® assay were correlated to results from assaying a venous blood sample spotted on one or more DPS cards from the subject. In some embodiments, Pearson's correlations are used to assess relationships between DPS samples and plasma from the subject. In some embodiments, Pearson's correlations are used to assess relationships between DPS samples from the subject and plasma samples from one or more subjects with a known amyloid β status. In some embodiments, the known amyloid β status is amyloid β positive. In some embodiments, the known amyloid β status is amyloid β negative.

In some embodiments, the detected protein level of each biomarker tested is used to assess a risk of the subject for developing cognitive decline as a consequence of neurodegeneration, traumatic brain injury (regardless of severity, including mild traumatic brain injury or concussion), cardiac arrest (or other causes of brain hypoxia), a neuroinflammatory condition, or a combination thereof. In some embodiments, the detected protein level of each biomarker tested is used to pre-screen and identify if the subject has elevated risk for developing cognitive decline prior to the subject undergoing a clinical evaluation. In some embodiments, a risk of the subject developing cognitive decline is determined prior to the subject presenting one or more behavioral symptoms associated with cognitive decline. In some embodiments, the detected protein level of each biomarker tested is used as a component in diagnosis of a neurodegenerative condition.

In some embodiments, the detected protein level of each biomarker tested is determined in at least two biological samples, each taken from the subject at different times. In some embodiments, a change in detected protein level of each biomarker tested over time is used to monitor a progression of a neurodegenerative condition. In some embodiments, a change in detected protein level of each biomarker tested over time is used to assess a response to a treatment in the subject for a neurodegenerative condition. In some embodiments, a change in detected protein level of each biomarker tested over time is used to assess an occurrence of an adverse effect during a treatment with an amyloid immunotherapy. In various embodiments, the adverse effect comprises amyloid related imaging abnormalities (ARIA). In some embodiments, the method further comprises sequentially monitoring an evolution of the adverse effect or sequentially monitoring a resolution of the adverse effect. In some embodiments, a change in detected protein level of each biomarker tested over time is used to monitor a progression of an acute traumatic brain injury. In some embodiments, a change in detected protein level of each biomarker tested over time is used to monitor a progression of an acute, mild traumatic brain injury. In some embodiments, a change in detected protein level of each biomarker tested over time is used to monitor a progression a concussion. In some embodiments, a change in detected protein level of each biomarker tested over time is used to assess severity of an acute traumatic brain injury. In some embodiments, a change in detected protein level of each biomarker tested over time is used to assess severity of a concussion. In some embodiments, a change in detected protein level of each biomarker tested over time is used to diagnose a concussion.

In some embodiments, the neurodegenerative condition comprises a tauopathy. In some embodiments, the tauopathy comprises Alzheimer's disease (AD) or Alzheimer's disease pathology or pathophysiology, preclinical AD, prodromal AD, AD dementia, Pick's disease, Niemann-Pick disease type C, frontotemporal dementia (FTD), frontotemporal lobar degeneration (FLD), chronic traumatic encephalopathy (CTE) or traumatic encephalopathy syndrome (TES), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, or a transgene-induced tauopathy. In some embodiments, the tauopathy comprises Alzheimer's disease.

In another aspect, the present disclosure provides a method of identifying if a subject is at risk for having or developing a neurodegenerative disease, the method comprising: a) obtaining a biological sample, wherein the biological sample comprises a blood sample derived from the subject; b) extracting proteins from the biological sample using a protein extraction reagent to obtain a protein-enriched extracted sample; c) performing an assay on the protein-enriched extracted sample, wherein the assay comprises determining a detected protein level of at least one of biomarkers: i) phosphorylated Tau-181 (pTau-181), ii) phosphorylated Tau-217 (pTau-217), iii) glial fibrillary acidic protein (GFAP), iv) neurofilament light chain (NFL), v) phosphorylated Tau-231 (pTau-231), vi) phosphorylated Tau-212 (pTau-212), vii) phosphorylated Tau-212+217 (pTau-217+), viii) brain-derived tau (BD-Tau), ix) 42-amino acid β amyloid (Aβ42) peptide, or x) 40-amino acid 1 amyloid (Aβ40) peptide; d) providing the detected levels of biomarkers to perform an evaluation step, wherein the evaluation step comprises comparing the provided detected levels of biomarkers in the protein-enriched extracted sample derived from the subject to threshold levels of biomarkers detected in a plurality of healthy subjects having normal cognitive function who have not been diagnosed with a neurodegenerative disease; and e) estimating a likelihood of the subject for having a neurodegenerative disease or developing a neurodegenerative disease, following completion of the evaluation step. In some embodiments, the blood sample comprises a capillary blood sample or a venous blood sample. In some embodiments, the blood sample comprises a capillary blood sample. In some embodiments, the blood sample comprises a capillary blood sample collected remotely. In some embodiments, the blood sample is a capillary blood sample. In some embodiments, the blood sample is a capillary blood sample collected remotely. In some embodiments, one or more DPS cards are used for the remote blood sample collection. In some embodiments, the blood sample is a venous blood sample.

In another aspect, the present disclosure provides a method for treating a neurodegenerative disease in a subject in need thereof, the method comprising: i) determining protein levels of one or more biomarkers in a biological sample by: a) obtaining a biological sample, wherein the biological sample comprises a blood sample derived from the subject; b) extracting proteins from the biological sample using a protein extraction reagent to obtain a protein-enriched extracted sample; and c) performing an assay on the protein-enriched extracted sample, wherein the assay comprises determining a detected protein level of at least one of biomarkers: i) phosphorylated Tau-181 (pTau-181), ii) phosphorylated Tau-217 (pTau-217), iii) glial fibrillary acidic protein (GFAP), iv) neurofilament light chain (NFL), v) phosphorylated Tau-231 (pTau-231), vi) phosphorylated Tau-212 (pTau-212), vii) phosphorylated Tau-212+217 (pTau-217+), viii) brain-derived tau (BD-Tau), ix) 42-amino acid β amyloid (Aβ42) peptide, or x) 40-amino acid 1 amyloid (Aβ40) peptide; and ii) administering a therapy to the subject. In some embodiments, the blood sample comprises a capillary blood sample or a venous blood sample. In some embodiments, the blood sample comprises a capillary blood sample. In some embodiments, the blood sample comprises a capillary blood sample collected remotely. In some embodiments, the blood sample is a capillary blood sample. In some embodiments, the blood sample is a capillary blood sample collected remotely. In some embodiments, one or more DPS cards are used for the remote blood sample collection. In some embodiments, the blood sample is a venous blood sample.

### Kits

Disclosed herein, in some embodiments, are kits comprising one or more DPS cards and instructions comprising one or more methods of the present disclosure. In some embodiments, the kit comprises a suitable lancet (e.g., a single-use safety lancet (Unistik 3, 30G)) for capillary blood sample collection on the one or more DPS cards. In some embodiments, the instructions describe applying a single hanging drop (˜50-60 μL) of blood from the finger-prick on the subject to each blood spot collection location on the card. In some embodiments, the instructions further comprise instructions to leave the DPS card with applied blood samples to dry at room temperature for 15 minutes. In some embodiments, the instructions further comprise instructions to ship the DPS card following the drying to a laboratory location for quantitative analysis of protein analytes from the sample.

Disclosed herein, in some embodiments, are kits comprising a protein extraction reagent described herein and instructions for use in a protein extraction and analysis protocol. In some embodiments, the instruction comprise a description of one or more methods described herein. In some embodiments, the instructions comprise instructions for incubating filters from one or more DPS cards with a protein extraction reagent for a period of 30 minutes at 37° C. shaking at 800 rpm. In some embodiments, the kit further comprises a quantification plate. In some embodiments, the instructions comprise instructions for depositing the analyte for detection into the quantification plate by centrifugation. In some embodiments, the instructions comprise instructions for using an ultra-sensitive protein detection system for quantifying a level of one or more selected protein biomarkers presenting the sample eluted from the DPS filters and into the quantification plate. In some embodiments, the instructions are for use of a Simoa® detection platform. In some embodiments, the instructions are for use of an MDS detection platform. In some embodiments, the instructions are for use of Lumipulse® immunoassay analysis platform. In some embodiments, the instructions are for use of a Elecsys Cobas® e801 immunohistochemistry detection platform. In some embodiments, the instructions are for use of an Ella Automated Immunoassay System platform. In some embodiments, the instructions are for use of an ARCHITECT Immunoassay System analysis platform. In some embodiments, the instructions are for use of an Atellica® Solution Immunoassay platform. In some embodiments, the instructions are for use of an ARGO™ HT System Immunoassay platform (Alamar). In some embodiments, the kit further comprises an antibody specific for detection of one or more selected neuro biomarkers. In some embodiments, the antibody specific for detection of one or more selected neuro biomarkers is a p-tau181 antibody, a p-tau212 antibody, a p-tau217 antibody, a p-tau217+ antibody, a p-tau-231 antibody, a total-tau antibody, a BD-tau antibody, a GFAP antibody, an NfL antibody, an Aβ40 antibody, an Aβ42 antibody, or an UCHL1 antibody.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the terms “a” or “an” means that “at least one” or “one or more” unless the context clearly indicates otherwise. The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in various embodiments, to A without B (optionally including elements other than B); in another embodiment, to B without A (optionally including elements other than A); in yet another embodiment, to both A and B (optionally including other elements); etc. The term “or” as used herein shall only be interpreted as indicating exclusive alternatives (i.e., “one or the other but not both”) when preceded by terms of exclusivity, “either,” “one of,” “only one of,” or “exactly one of”.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, “either,” “one of,” “only one of,” or “exactly one of.” “Consisting essentially of,” when used in the claims, shall have its ordinary meaning as used in the field of patent law.

The term “about” as used herein when referring to a measurable value such as an amount, a temporal duration, and the like, is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value, as such variations are appropriate to perform the disclosed methods.

As used herein, the term “kit” refers to a set of components provided in the context of a system for diagnosing a subject having, or suspected of having, a neurological or neurodegenerative condition. Such delivery systems may include, for example, systems that allow for storage, transport, or delivery of various diagnostic reagents (e.g., one or more DPS cards and a finger-prick lance) and/or supporting materials (e.g., buffers, media, written instructions for performing sample collection and processing, etc.) from one location to another. For example, in some embodiments, kits include one or more enclosures (e.g., boxes) containing relevant reaction reagents and/or supporting materials.

As used herein, the terms “subject” or “subjects” or “individual” or “individuals” refer to humans and the terms may be used interchangeably.

As used herein a composition that is “consisting essentially” of the recited components is a composition that only has the recited elements as active ingredients, but can comprise other non-active components that do not appreciably modify the function or activity of the recited components. Any list disclosed herein that is recited as “comprising” can be recited as “consisting essentially,” to exclude non-recited protein extraction reagent components.

The abbreviations used herein have their conventional meaning within the chemical and biological arts.

As used herein, the term “diagnosed” means having been subjected to a physical examination by a person of skill, for example, a physician, and found to have a condition that can be diagnosed or treated by the compounds, compositions, or methods disclosed herein. In some embodiments of the disclosed methods, the subject has been diagnosed with a need for treatment of a disorder having neurodegeneration. As used herein, the phrase “identified to be in need of treatment for a disorder,” or the like, refers to selection of a subject based upon need for treatment of the disorder. It is contemplated that the identification can, in some embodiments, be performed by a person different from the person making the diagnosis. It is also contemplated, in further embodiments, that the administration of a diagnosis can be performed by one who previously or subsequently performed the administration of a treatment disclosed herein.

The section headings used herein are for organizational purposes only and are not to be construed as limiting the subject matter described.

### Examples

The following examples are included for illustrative purposes only and are not intended to limit the scope of the invention.

### Example 1: Biomarker Correlations from Venous Blood Extract from Dry Drop and Blood Plasma

In this example, biomarker correlations obtained from venous blood extracted from dry drop blood using different devices were compared with blood plasma. Biomarkers for Alzheimer's disease such as p-tau181, p-tau217, p-tau231, NfL, GFAP, Aβ42 and Aβ40 were investigated.

### Methods

Venous blood collected via venipuncture in K²EDTA tubes, was spotted onto different dried blood drop cards (or dried plasma spot cards) and extracted using the in-house extraction protocol. Each dried blood drop card has two separated filters. These filters can separate plasma from cells and platelets. Each filter was treated with either the “High-Performance Protein Extraction Buffer” or the “Medium Performance Protein Extraction Buffer” in order to produce one or more protein-enriched extracted sample used to detect and quantify specific biomarkers. Each separate filter was subjected to protein extraction using a specific extraction buffer to create a protein-enriched extracted sample representing an extent and utility of protein extraction specific to, and that can be optimized for, assay of one or more specific biomarkers. The same samples were processed according to standardized methods to obtain EDTA plasma. Both the extracted samples from the dried blood drop card and the plasma extracted from venous blood collection were analyzed using p-tau181 (Quanterix), p-tau217 (ALZpath), p-tau231 (University of Gothenburg). In addition, NfL, GFAP, Aβ42 and Aβ40 (Quanterix multiplex) were also analyzed.

The “High-Performance Protein Extraction Buffer” was used for phosphorylated protein extraction and later detection and quantification. The in-house extraction protocol with the “High-Performance Protein Extraction Buffer” was developed to detect and quantify p-tau markers such as i) pTau-181, ii) pTau-217, v) pTau-231, vi) pTau-212, vii) pTau-217+, or viii) BD-Tau. The composition of the “High-Performance Protein Extraction Buffer” comprises 20 mM Tris, 137 mM NaCl, 1% BSA, 0.05% ProClin™ 300, 0.10 polysorbate nonionic surfactant, pH 9.0. Optionally, 5 mM EDTA can be added to the “High-Performance Protein Extraction Buffer.” The 0.1% polysorbate nonionic surfactant used in this example was 0.10% Tween 20.

The in-house extraction protocol with the “Medium Performance Protein Extraction Buffer” was developed to test other biomarkers such as i) glial fibrillary acidic protein (GFAP), ii) neurofilament light chain (NFL), iii) 42-amino acid 3 amyloid (Aβ42) peptide, or iv) 40-amino acid 3 amyloid (Aβ40) peptide. The composition of the “Medium Performance Protein Extraction Buffer” comprises Phosphate, NaCl, KCl, BSA, TRU Block™, Tween 20, ProClin™ 300, and EDTA, pH 7.4. In some instances, the composition of the “Medium Performance Protein Extraction Buffer” comprises Phosphate, NaCl, KCl, BSA, MgCl₂, Dextrose, Urea, 1.0% TRU Block™, ProClin™ 300, pH 7.4. In some instances, TRU Block™ immunoassay blocker is present at about 10 μg/mL.

The “Low Performance Protein Extraction Buffer” was also tested to compare the efficacy and utility of protein extraction using either a High Performance Protein Extraction Buffer or a Medium Performance Protein Extract Buffer to other methods. The composition of the “Low Performance Protein Extraction Buffer” comprises phosphate, NaCl, KCl, BSA, Tween 20, ProClin™ 300, and EDTA, TRU Block™, pH 7.4. These compositions were tested at different concentrations.

To proceed with biomarker quantification, the first step was to extract the filters from the dried blood drop cards and place them into the designated extraction devices comprising membrane embedded chamber. The selection of these devices was based on strong correlations with plasma values and suitability for high throughput preparation. Other options were discarded for low protein retention, weaker correlations with EDTA plasma or difficulties for high throughput. Next, 150 μL of the extraction buffer were added to each well within the extraction device, and the samples were incubated for 30 minutes at 37° C. shaking at 800 rpm. Various buffers have been explored to optimize sample extraction from the cards, ensuring compatibility with different immunoassays and analyte target quantification methods. Following the incubation period, the extraction devices, along with the biomarker quantification plate, were centrifuged so the sample was directly deposited in the quantification plate, and was ready to be measured.

Extraction devices for high performance used in this example was spin buckets (Sigma-Aldrich, ref Q3258). For medium performance, Fritted and unfritted deep 96-well plates (ThermoScientific, ref 278011 and 278012) were used. Various other filters are used with the method described in this example using the high performance extraction buffer for efficient, functional, and informative protein extraction to obtain a protein-enriched extracted sample optimized for assaying particular neuro biomarkers.

The method described herein has been proved compatible with several assays such as p-tau217 (ALZpath, Simoa platform), NfL & GFAP (Quanterix 2NPB, Simoa platform), p-tau181 (Quanterix, Platform), and p-tau181 (ADx, Platform). Additionally, the method described herein can be used with Meso Scale Discovery (MSD), Lumipulse (Fujirebio), Elecsys (Roche), and Mass Spectrometry.

### Results

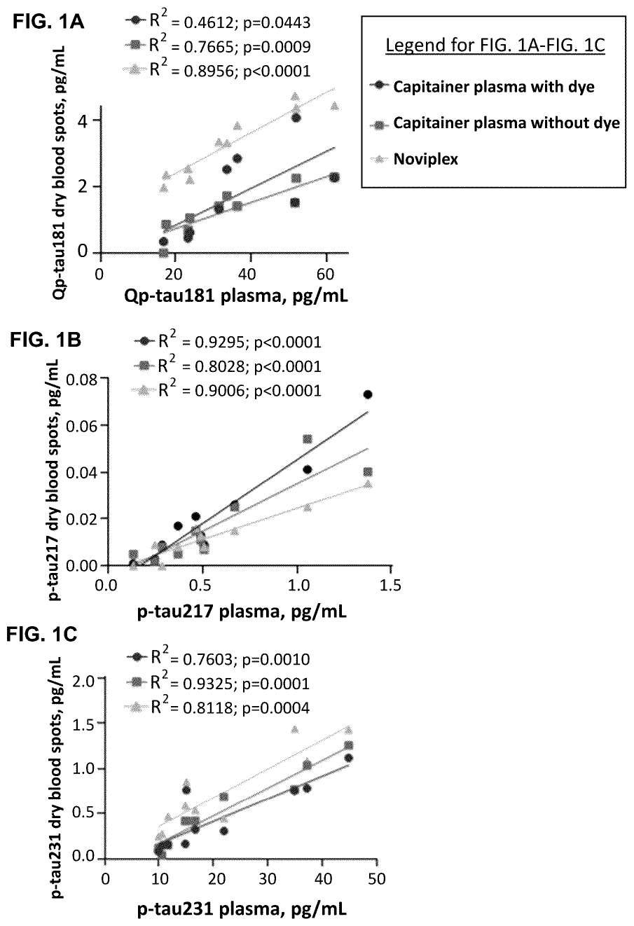

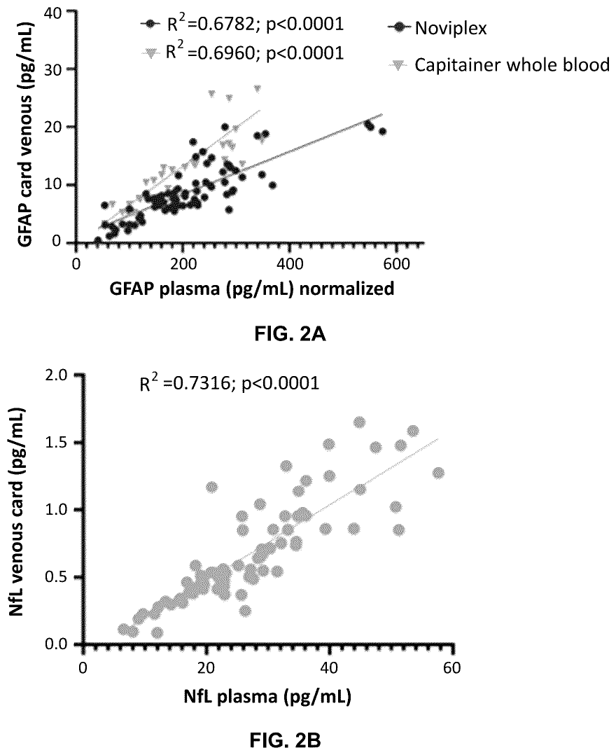

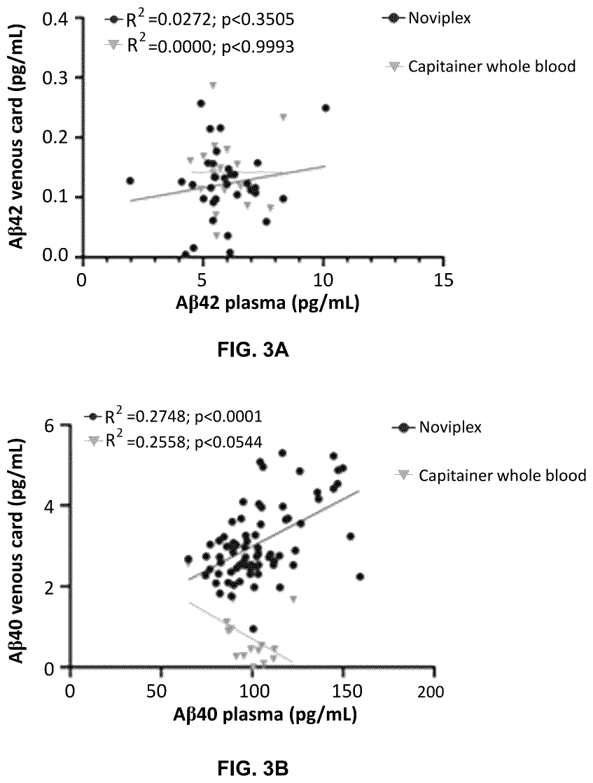

The preliminary results indicate that all devices, both dried blood drop cards and standard methods of venous blood collection, work with the in-house extraction protocol. Neuro biomarkers were detected and quantified. The measurements of p-tau181, p-tau217, p-tau231, GFAP and NfL in spotted venous blood obtained from dried blood drop cards exhibit strong correlation with the measurements in plasma obtained from venipuncture blood collection. This is shown in FIG. 1A-FIG. 1C correlating p-tau measurements derived from samples collected using dried blood drop cards to p-tau measurements derived from samples from plasma isolated from venous blood and in FIG. 2A-FIG. 2B correlating GFAP and NfL measurements derived from samples collected using dried blood drop cards to respective GFAP and NfL measurements derived from samples from plasma isolated from venous blood. As shown in FIG. 3A-FIG. 3B, the measurement and correlation of Aβ42 and Aβ40 was not consistently observed (FIG. 3A for Aβ42 R²=0.0272 for Noviplex and R²=0.0000 for Capitainer whole blood; FIG. 3B for Aβ40 R²=0.2748 for Noviplex and R²=0.2558 for Capitainer whole blood). The x-axis represents samples obtained from standard method of blood plasma collection. The y-axis represents extracted samples collected using dried blood drop cards, such as Noviplex or Capitainer whole blood.

Graphpad Prism 9 was used for statistical analysis. Data were inspected for normality and non-parametric tests used for non-normal distributions. Pearson's correlations were used to assess relationships between DPS samples and plasma. As shown in FIG. 1A, strong positive correlations were present for p-tau181 (Quanterix p-tau 181 (Qp-tau181)) comparing dry blood spot and plasma samples using various microsampling collection devices (R²=0.4612; p=0.0443 for Capitainer plasma with dye/R²=0.7665; p=0.0009 for Capitainer plasma without dye/R²=0.8956; p<0.0001 for Noviplex). As shown in FIG. 1B, strong positive correlations were present for p-tau217 comparing dry blood spot and plasma samples using various microsampling collection devices (R²=0.9295; p<0.0001 for Capitainer plasma with dye/R²=0.8028; p<0.0001 for Capitainer plasma without dye/R²=0.9006; p<0.0001 for Noviplex).

As shown in FIG. 1C, strong positive correlations were present for p-tau231 comparing dry blood spot and plasma samples using various microsampling collection devices (R²=0.7603; p=0.0010 for Capitainer plasma with dye/R²=0.9325; p=0.0001 for Capitainer plasma without dye/R²=0.8118; p=0.0004 for Noviplex). These results indicate strong correlation with results obtained from capillary dry blood spots to results obtained from a standard biomarker assay source (e.g., plasma isolated from a venous blood sample).

As shown in FIG. 2A, strong positive correlations were present for GFAP comparing dry blood spot from venous samples and plasma samples from venous blood sample using various microsampling collection devices (R²=0.6782; p<0.0001 for Noviplex/R²=0.6960; p<0.0001 for Capitainer whole blood). As shown in FIG. 2B, strong positive correlations were present for NfL comparing dry blood spot from venous samples and plasma samples from venous blood sample using a microsampling collection device (R²=0.7316; p<0.0001 for Noviplex).

As shown in FIG. 3A, a positive correlation was identified for Aβ40 comparing dry blood spot from venous samples and plasma samples from venous blood sample using a microsampling collection device (R²=0.2558; p<0.0001 for Noviplex). As shown in FIG. 3B, a negative correlation nearing significance was identified for Aβ40 comparing dry blood spot from venous samples and plasma samples from venous blood sample using a different microsampling collection device (R²=0.2558; p<0.0544 for Capitainer whole blood).

Overall, these results show that detection of p-tau181, p-tau217, p-tau231, GFAP and NfL in spotted venous blood obtained from dried blood drop cards using in-house methods and extraction buffers correlates with the measurements in blood plasma obtained from venipuncture blood collection.

### Example 2: Biomarker Correlations from Capillary and Venous Blood Extracted from Noviplex Cards and Blood Plasma

In this example, biomarker correlations obtained from capillary and venous blood extracted from dried blood drop cards, such as Noviplex cards, as dried drop blood format were compared with blood plasma obtained from capillary and venous blood collection using standard methods.

### Methods

Capillary and venous blood was spotted in Noviplex cards and extracted using the in-house extraction protocol and protein extraction buffers as described in Example 1. Noviplex cards using dried plasma spot technology from Shimadzu are now referred to as Telimmune Plasma Separation Cards. Telimmune Plasma Separation Cards utilize simultaneous cell partition technology to eliminate metabolic effects associated with standard liquid phlebotomy samples. Plasma is automatically fractionated from a whole blood specimen (e.g., a finger-prick capillary blood drop) collected on the card. Use of these cards delivers approximately 3 μL of plasma per disk (two disks per card). Collection disks may be air dried to aid in stabilizing the sample. This enables inexpensive and safe transport for later in vitro analysis of neuro biomarkers extracted from the plasma samples collected on the disks. Capitainer® dried blood spot microsampling cards operate in a similar manner to Telimmune Plasma Separation Cards during collection of a blood spot sample and fractionation of whole blood to collect plasma on disks (two disks per card). The same samples were processed using standard methods to obtain plasma. Both the extracted samples from dried blood drop cards and the plasma were analyzed using p-tau181 (Quanterix), p-tau217 (ALZpath), p-tau231 (University of Gothenburg). In addition, NfL and GFAP (Quanterix 2-Plex) were also analyzed.

### Results

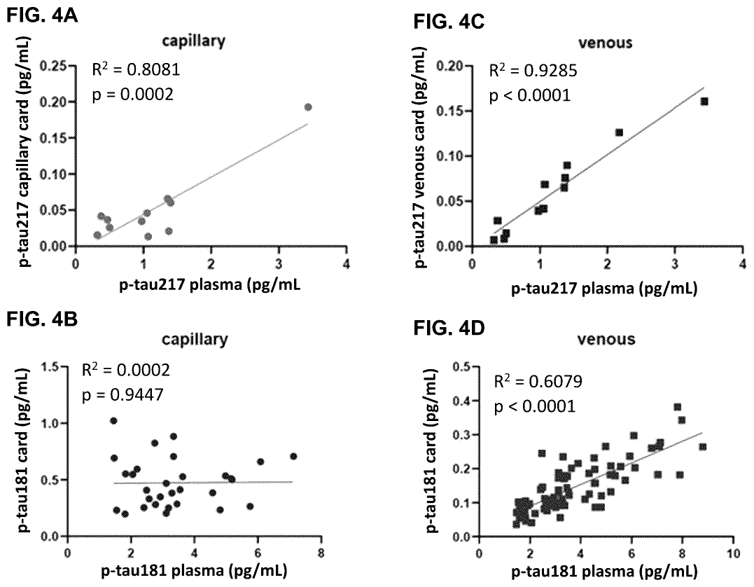

The preliminary results indicate that all devices, both dried blood drop cards and standard methods of capillary and venous blood collection, work with the in-house protocol. The measurements of p-tau217 in spotted capillary and venous blood obtained from dried blood drop cards correlate with the measurements in plasma obtained from standard method, as shown in FIG. 4A and FIG. 4C. For the measurements of p-tau217 (in FIG. 4A), the correlation in spotted capillary blood obtained from dried blood drop cards compared to plasma obtained from standard method is observed (R²=0.8081; p<0.0001). For the measurements of p-tau217 (in FIG. 4C), the correlation in spotted venous blood obtained from dried blood drop cards compared to plasma obtained from standard method is observed (R²=0.9285; p<0.0001). The measurements of p-tau181 in spotted venous blood obtained from dried blood drop cards correlate with the measurements in plasma obtained from standard method, as shown in FIG. 4D. The measurements of p-tau181 in spotted capillary blood obtained from dried blood drop cards did not demonstrate a correlation with the measurements in plasma obtained from standard method, as shown in FIG. 4B (R²=0.0002; p=0.9447). FIG. 4D), the correlation in spotted venous blood obtained from dried blood drop cards compared to plasma obtained from standard method is observed (R²=0.6079; p<0.0001). For the measurements of p-tau181 (in FIG. 4B), the correlation in spotted venous blood obtained from dried blood drop cards compared to plasma obtained from standard method is observed (R²=0.6079; p<0.0001)

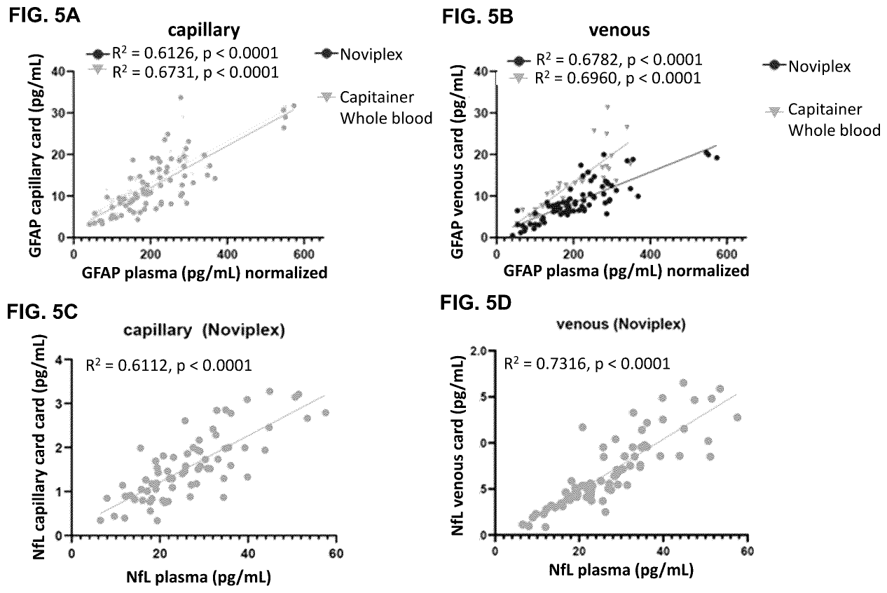

The measurements of GFAP and NfL in spotted capillary and venous blood obtained from dried blood drop cards correlate with the measurements in plasma obtained from standard method, as shown in FIG. 5A-FIG. 5D. For the measurements of GFAP, the correlation in spotted capillary blood obtained from dried blood drop cards compared to plasma obtained from standard method is observed (R²=0.6126; p<0.0001 using Noviplex and R²=0.6731; p<0.0001 using Capitainer whole blood (in FIG. 5A)). For the measurements of GFAP, the correlation in spotted venous blood obtained from dried blood drop cards compared to plasma obtained from standard method is observed (R²=0.6782; p<0.0001 using Noviplex and R²=0.6960; p<0.0001 using Capitainer whole blood (in FIG. 5B)). For the measurements of NfL, the correlation in spotted capillary blood obtained from dried blood drop cards compared to plasma obtained from standard method is observed (R²=0.6112; p<0.0001 using Noviplex (in FIG. 5C)). For the measurements of NfL, the correlation in spotted venous blood obtained from dried blood drop cards compared to plasma obtained from standard method is observed (R²=0.7316; p<0.0001 using Capitainer whole blood (in FIG. 5D)).

Overall, these results show that detection of p-tau181, p-tau217, p-tau231, GFAP and NFL in capillary and venous blood extracted from Noviplex cards using in-house methods and extraction buffers correlates with the measurements in blood plasma obtained from standard method of capillary and venous blood collection.

### Example 3: Biomarker Comparison with Cerebrospinal Fluid Samples

Detection of amyloid status from cerebrospinal fluid (CSF) sample is used as a gold standard measurement for Alzheimer's disease pathology. Relationship of amyloid status and biomarkers obtained from dried blood drop collection using methods and devices described in the previous examples was investigated in this example.

In this example, CSF samples obtained from subjects were collected and tested for the amyloid status. Venous blood collection using dried blood drop cards was also collected from the same subjects. The following markers: NfL, GFAP, and p-tau181 using methods and buffers described in the Example 1 were measured.

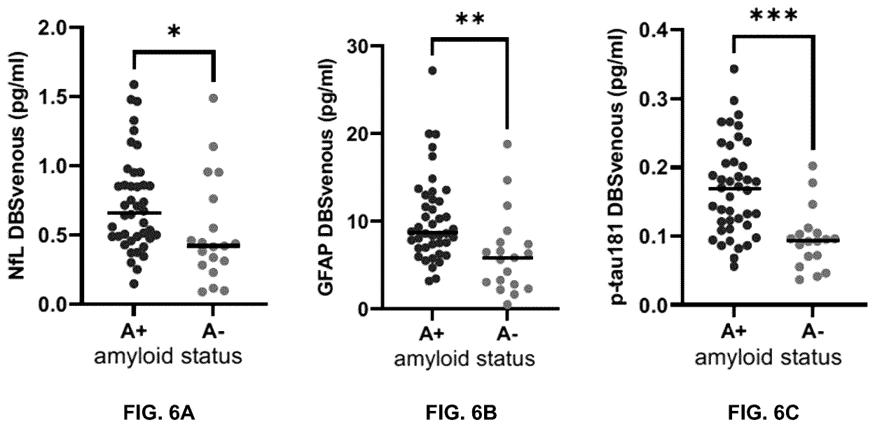

FIG. 6A-FIG. 6C depict that amyloid positive status (A+) in a subject (determined from amyloid measurements in CSF samples) show higher level of NfL, GFAP, and p-tau181 obtained from dried blood sample from venous blood from a subject when compared amyloid negative status (A−) in a subject (determined from amyloid measurements in CSF samples). DPS venous results plotted on the y-axis indicate results for dried blood sample spotted on the card devices obtained from venous blood (* p<0.05; ** p<0.01; *** p<0.001).

The graph in FIG. 6A shows significantly elevated NfL detected from dried blood sample from venous blood in A+ subjects compared to A− subjects. The graph in FIG. 6B shows significantly elevated GFAP detected from dried blood sample from venous blood in A+ subjects compared to A− subjects. The graph in FIG. 6C shows significantly elevated p-tau181 detected from dried blood sample from venous blood in A+ subjects compared to A− subjects.

Overall, these results suggest that biomarkers obtained from dried blood drop cards correlate with amyloid status detected from CSF. Results from the methods and buffers described herein are confirmed with accuracy and specificity. Furthermore, the levels of these biomarkers from subjects with unknown status of Alzheimer's disease can also be compared to the reference levels, such as the levels of biomarkers obtained from amyloid positive status or from Alzheimer's patient cohorts.

### Example 4: Ultrasensitive Quantification of Neurodegeneration Biomarkers Neurofilament Light and Glial Fibrillary Acidic Protein in Finger-Prick Blood

The aim of this experiment is to investigate if NfL and GFAP can be accurately measured in finger-prick blood using dried plasma spot (DPS) cards (or dried blood drop card).

### Methods

Fifty-five patients (49 with dementia; 6 with TBI) and eighteen healthy volunteers underwent finger-prick and venous sampling using DPS cards and aligned plasma sampling. NfL and GFAP were quantified using a Single molecule array (Simoa) assay and correlations between plasma and DPS cards were assessed.

Patient samples were analyzed across several research studies. People with dementia were recruited from an ongoing longitudinal home-monitoring cohort study ‘MINDER’ run by the UK Dementia Research Institute (DRI) Care Research and Technology Centre at Imperial College London. A further group of patients with dementia and healthy volunteers were recruited from an ongoing trial of noradrenergic add-on therapy with extended-release Guanfacine in Alzheimer's disease (NorAD). Patients were no longer taking the investigational medicinal products at the time of blood sample collection. Patients within ten days of acute moderate-severe TBI were recruited from the major trauma centre at Imperial College Healthcare NHS Trust, with severity classified by the Mayo severity classification (Malec et al., 2007). All participants provided written informed consent. Research ethics approvals were granted by the relevant local committees.

Venous blood was sampled peripherally and collected using ethylenediaminetetraacetic acid-coated tubes for plasma. After 30 minutes at room temperature, samples were centrifuged at 2500 g at 4° C. for 20 minutes, then transferred into 1.4 mL aliquots, and frozen at −80° C. Matched DPS were collected using Telimmune™ Plasma Prep Duo Cards, a micro-sampling tool for plasma collection (Shimadzu, JP) (Kim et al., 2013). The DPS were produced from venous blood sampled by venipuncture and from a paired finger-prick blood sample, obtained using a single-use safety lancet by a clinician (Unistik 3, 30G). Two drops of venous and finger-prick blood (approximately 60 μL) was spotted onto each Telimmune Duo card, producing two DPS per card. The cards were left to dry at room temperature for 15 min. They were then placed back in their original foil pouches containing a silica desiccant and stored at room temperature until analysis (up to 15 months). Cards were stored at room temperature with samples analyzed a mean of 9.7 months (SD 2.5) prior to analysis.

NfL and GFAP concentrations were measured using a 2-plex assay on a Quanterix Single molecule array (Simoa) HD-X analyzer in three aligned samples for each individual: Tellimune finger-prick DPS, Tellimune venous DPS, and matched plasma samples. Calibrators were run in duplicates and obvious outlier calibrator replicates were masked before curve fitting. Plasma samples were diluted 4-fold and run in singlicates. Results have been compensated for the dilution. Tellimune cards were eluted in an in-house sample buffer and run in singlicates. Two QC levels were run in duplicates in the beginning and the end of each run. In relation to NfL, for a QC sample with a concentration of 16.1 μg/mL, repeatability was 4.2% and intermediate precision was 4.2%; for a QC sample with a concentration of 91.2 μg/mL, repeatability was 5.2% and intermediate precision was 5.2%. In relation to GFAP, for a QC sample with a concentration of 117 μg/mL, repeatability was 5.8% and intermediate precision was 10.9%; for a QC sample with a concentration of 337 μg/mL, repeatability was 9.7% and intermediate precision was 14.7%.

Quantification within a Tellimune card failed in one healthy volunteer and one patient with AD, due to technical error. Results were not reported for Tellimune cards in 4 healthy volunteers where the GFAP concentration was below the lower limit of quantification (LLOQ), and in one card where the NfL concentration was below the LLOQ. Participants with dementia underwent the Addenbrooke's Cognitive Examination (Mioshi et al., 2006) (version III) on the day of the blood test, except in a subset where this was linearly imputed from the Alzheimer's Disease Assessment Scale-Cognitive Subscale-14, within 6 months of the blood test according to standard approaches (Caro et al., 2002; Rosen et al., 1984).

R version 4.2.2 (2022-10-31)/Rstudio 2022.12.0+353 were used for statistical analysis. Data were inspected for normality and non-parametric tests used for non-normal distributions. Chi-squared test was used to assess sex differences by group, and the Kruskal test for age, using Dunn's post hoc test (Benjamini-Hochberg method for P value adjustment for multiple comparisons). Pearson's correlations were assessed relationships between DPS samples and plasma. Linear mixed effects models (R package nlme) accounting for individual (random effect) were used to test how biomarker concentrations varied depending on biofluid (e.g., across plasma, finger-prick DPS and venous DPS). Linear regression was used to test the variation in plasma concentration by participant group, with concentration log-transformed (due to non-linearity) and with age as a nuisance regressor.

### Results

A total of 46 patients with dementia, 4 with acute TBI and 19 healthy volunteers underwent neurodegeneration biomarker assessment (Table 1). Of the patients, AD dementia was the most common diagnosis in n=42 (91% of the dementia group). There was no significant difference in sex by group (P>0.05). There was no significant difference in age between TBI and healthy controls, but patients with dementia were significantly older than healthy controls (Z=2.9, P=0.005). Hence, age was included as a covariate in subsequent regressions assessing biomarkers by group. Patients with dementia were typically 5.8 years following diagnosis (mean, SD 3.6). The average score on the Addenbrooke's Cognitive Assessment was 64.9 (SD 18.6), where the maximum possible score is 100 and a mild/moderate-severe cut-off of 61 points is used (Giebel & Challis, 2017). Four patients with acute moderate-severe TBI were assessed within ten days of injury, all of whom had focal acute trauma-related pathologies on CT. Demographics including age and sex, by group, are described in detail in Table 1.

TABLE 1
Demographics and clinical characteristics.

| | Healthy volunteers | | Acute TBI | | Dementia | |
|---|---|---|---|---|---|---|
| N | 19 | | 4 | | 46 | |
| Age, mean (SD) | 56.6, | (22.4) | 30.2, | (6.4) | 76.4, | (7.5) |
| Sex, male, N (%) | 10 | (52.6%) | 3 | (75.0%) | 26 | (56.5%) |

| Clinical | — | Moderate-severe TBI (n = 4); | ACE total score mean 64.9 |
|---|---|---|---|
| characteristics | | time since injury mean 4.3 | (SD 18.6); years since |
| | | days (SD 4.9); focal traumatic | diagnosis mean 5.8 (SD 3.6); |
| | | CT abnormalities (n = 4); | aetiology AD (N = 42), FTD |
| | | aetiology: fall (n = 2), RTA | (N = 1), DLB (N = 1), VaD |
| | | (n = 2) | (N = 1) |

Demographics and clinical characteristics of study population. TBI=traumatic brain injury; TBI severity indicated by Mayo classification; focal traumatic abnormalities defined CT findings including any of extradural haematoma, subdural haematoma, subarachnoid blood, contusion. Dementia severity indexed usingAddenbrooke's Cognitive Assessment (ACE). AD: Alzheimer's dementia; FTD: frontotemporal dementia; DLB: dementia with Lewy Bodies; VaD: vascular dementia.

Concentrations of fluid biomarkers were quantified across plasma, venous DPS and finger-prick DPS (Table 2). First, using linear mixed effects modelling (with subject as a random effect), the differences in each biomarker's concentration by sample type were clarified. GFAP concentrations were substantially lower on DPS, for both finger-prick (reduced by 99.4% [95% CI 99.3-99.5]) and venous blood (by 99.0% [98.9-99.2]), compared with plasma. Similarly, for NfL, DPS concentrations were significantly lower on both finger-prick (by 99.3% [99.2-99.5]) and venous samples (−99.1% [98.8-99.2]) versus plasma.

TABLE 2
Fluid biomarker concentrations in healthy controls and patients.

| | | Healthy controls | | Acute TBI | | Dementia | |
|---|---|---|---|---|---|---|---|
| GFAP concentration, | Plasma | 125 | (146.2, n = 19) | 1802.5 | (3847.5, n = 4) | 311.5 | (216.8, n = 46) |
| pg/ml, median (IQR, N) | Finger-prick | 0.8 | (1, n = 17) | 4.5 | (10.1, n = 4) | 1.7 | (1.5, n = 32) |
| | Venous | 1.7 | (1, n = 14) | 9.7 | (22.9, n = 4) | 2.6 | (2.1, n = 44) |
| NfL concentration, | Plasma | 10.2 | (15.7, n = 19) | 106.2 | (204.4, n = 4) | 29 | (16.4, n = 46) |
| pg/ml, median (IQR, N) | Finger-prick | 0.1 | (0.1, n = 16) | 0.8 | (1.2, n = 4) | 0.2 | (0.2, n = 32) |
| | Venous | 0.2 | (0.1, n = 17) | 1.2 | (2.4, n = 4) | 0.2 | (0.2, n = 44) |

Biomarker concentrations in different bio-fluids. TBL traumatic brain injury; NfL: neurofilament light; GFAP: glial fibrillary acidic protein; IQR: interquartile range.

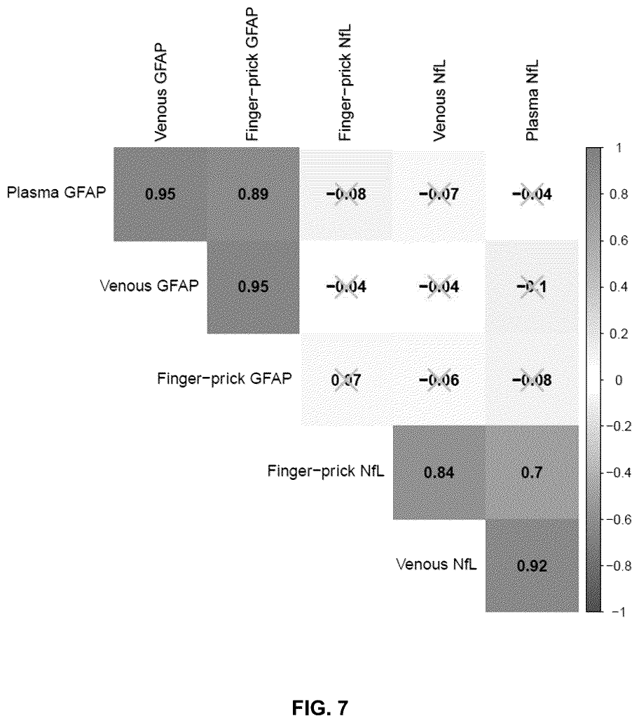

Next, the biomarker correlations between plasma and both finger-prick and venous DPS samples were tested, by individual (Pearson's correlation, see FIG. 7). Strong positive correlations were present for GFAP (R=0.89; P<0.001) and NfL (R=0.70; P<0.001) using finger-prick. Correlations between plasma samples and venous blood on DPS showed a stronger relationship (GFAP R=0.95; NfL R=0.92; P<0.001).

Next, the separation of the patient groups using different fluid samples (e.g., plasma, finger-prick and capillary DPS) was tested. Gold-standard plasma GFAP concentrations were significantly raised in both acute TBI (1280% [548-2830], P<0.001) and dementia (by 53.1% [1.45-131], P=0.04), compared with healthy controls. On finger-prick DPS testing, GFAP was significantly raised only in the TBI group (by 610% [163-1820], P<0.001) and likewise on venous DPS testing, significant elevation was only seen after TBI (475% [111-1470], P<0.001).

Plasma NfL was significantly increased in both acute TBI (1350% elevation [95% CI 655-2680, P<0.001] and dementia (67.3% increase [17.0-139], P=0.005) compared with healthy controls. On finger-prick NfL testing, concentrations were significantly elevated in acute TBI (939% higher [95% CI 266-2850]P<0.001) but a dementia-associated increase was not significant, versus controls. This was also the case on venous DPS NfL testing where elevation was significant after TBI (increase by 332% [90-881]) but not in dementia.

In FIG. 7, Pearson's correlations shown between neurofilament light (NfL) and glial fibrillary acidic protein (GFAP) tested in different fluids. Venous and finger-prick samples are using dried plasma cards (DPS). Correlation coefficients are shown in black text and indicated by gradient-bar, with darker color for perfect positive correlation. Insignificant correlations at a threshold of P>0.05 are shown crossed-out. All of the non-crossed correlations were highly significant at P<0.001

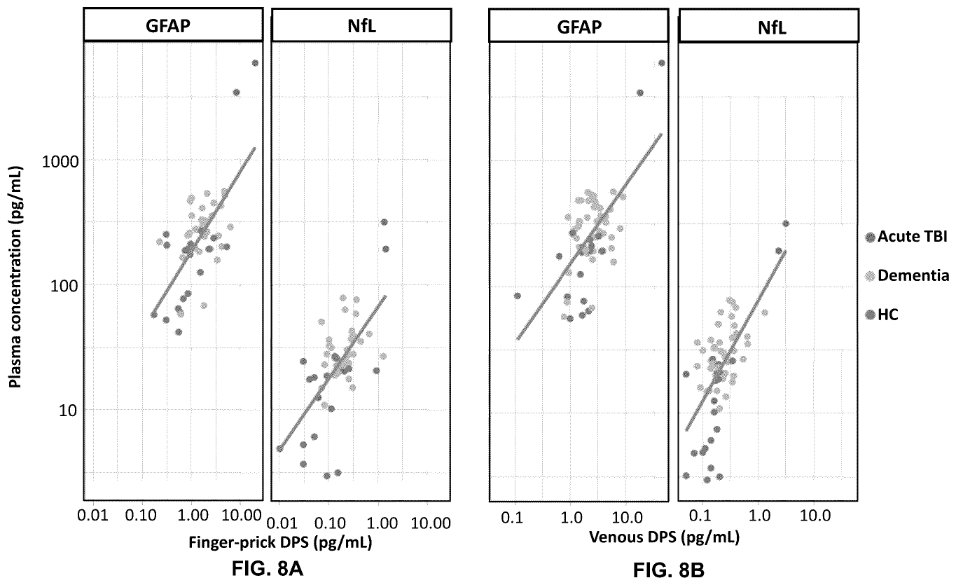

FIG. 8A-FIG. 8B show relationships of plasma biomarker concentrations to finger-prick and venous dried plasma spot measurements. Correlation between gold-standard plasma sampling (Y-axis) and finger-prick samples (X-axis) using dried plasma spots for glial fibrillary acidic protein (GFAP, left panel in FIG. 8A) and neurofilament light (NfL, right panel in FIG. 8A). Correlation between gold-standard plasma sampling (Y-axis) and venous samples (X-axis) using dried plasma spots for glial fibrillary acidic protein (GFAP, left panel in FIG. 8B) and neurofilament light (NfL, right panel in FIG. 8B). Each participant is represented by a single point, with patient/control group is indicated by shade of dot. Regression line is shown with 95% confidence interval. Groups of subjects having acute traumatic brain injury (Acute TBI), Dementia, or healthy volunteers as controls (HC) were tested and the results graphed in FIG. 8A-FIG. 8B.

Additional methods for use in evaluating the precision of Alzheimer's disease (AD) and neurodegeneration biomarker measurements from venous dried plasma spots for the diagnosis and monitoring of neurodegenerative diseases in remote settings are described in Huber H, et al. Biomarkers of Alzheimer's disease and neurodegeneration in dried blood spots—A new collection method for remote settings. Alzheimers Dement. 2024 Apr. 20(4):2340-2352; which is hereby incorporated by reference for the methods used to evaluate neurodegeneration biomarker measurements from venous dried plasma spot samples collected using DPS sampling cards.

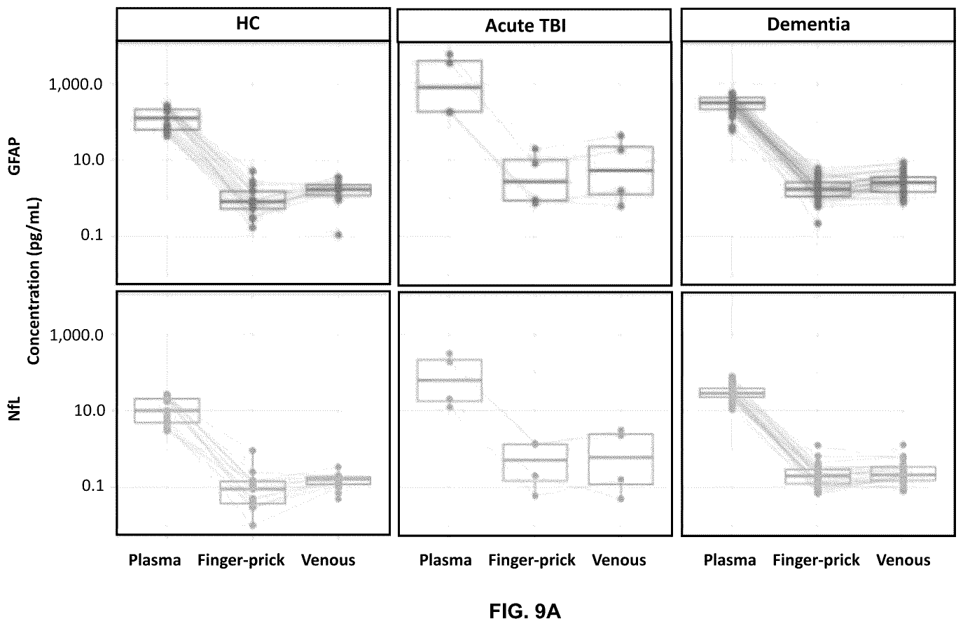

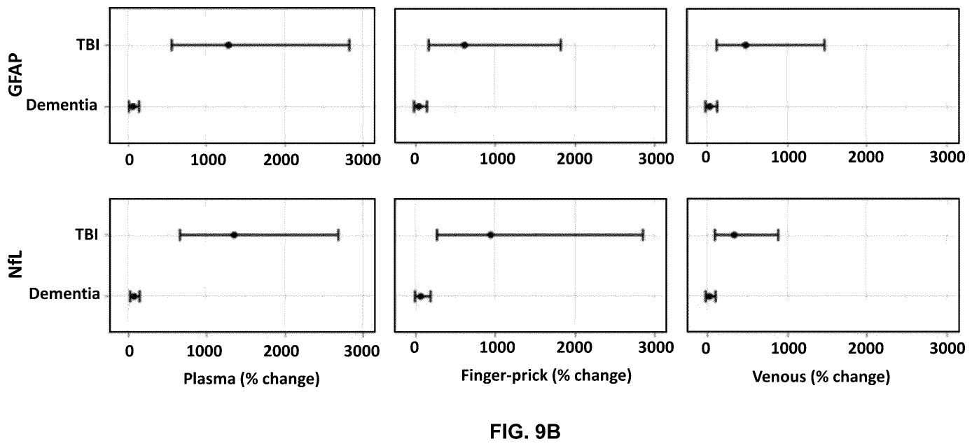

FIGS. 9A-9B shows variation in biomarker concentration by specimen and by participant group. FIG. 9A shows Group-wise concentrations of GFAP (top row) and NfL (lower row). Boxplots show median and interquartile ranges. Single data points shown, connected by lines to indicate same individual's data, with rank order largely preserved. FIG. 9B shows Forest plot showing % change in concentration of biomarkers by group (compared with healthy controls) in regression model for log(biomarker concentration. Point estimate shown by dot with 95% confidence intervals around estimate. Groups of subjects having acute traumatic brain injury (Acute TBI), Dementia, or healthy volunteers as controls (HC) were tested and the results graphed in FIG. 9A-FIG. 9B.

In this example, strong correlations between finger-prick blood sampling and formal plasma quantification of GFAP and NfL in a clinical cohort were observed, comprising patients with dementia and TBI, as well as healthy volunteers. The results show that GFAP has been successfully and accurately quantified using finger-prick DPS, and in patients with dementia or TBI. Clear differentiation of the acute TBI group using finger-prick GFAP and NfL was observed, as well as a non-significant trend towards separation in the dementia group.

Results show that concentrations were substantially elevated in plasma compared with DPS, by around 100 times. Further, these results show that venous blood applied to the DPS cards correlated more strongly with plasma than finger-prick blood and that finger-prick blood sampling using DPS has a range of advantages over traditional approaches. GFAP and NfL can be accurately quantified using finger-prick blood sampling with DPS cards. Overall, this technique has large potential for impact across a range of neurological disorders such as dementia and TBI, potentially widening access to diagnosis, disease monitoring and outcome assessment. Furthermore, the methods and compositions of buffer described herein can be used for analysis of other biomarkers, such as disease-specific molecules (e.g., phosphorylated tau in Alzheimer's Disease) and other neurological diseases (e.g., inflammatory diseases such as multiple sclerosis).

### Example 5: Suggested Workflow from Home Collection to Laboratory Analysis

Based on results from the previous examples, methods to collect blood samples can be done remotely on patients while still providing accuracy and specificity of the biomarkers. This remote collection from a finger-prick capillary blood sample can be collected by the patient themselves or by another individual (e.g., a caregiver, a household member, or an on-site healthcare professional). This less invasive blood collection procedures provides an alternative method of blood collection and biomarker detection. The in-house extraction protocol is highly convenient to implement. The cards intended for spotting can be provided together with the necessary reagents for the extraction of the dry drop, as well as the immunoassay reagents. Upon receipt of the spotted card, the laboratory can proceed to extract the samples using the provided extraction reagents and perform the corresponding immunoassay for the target analyte in a straightforward manner.

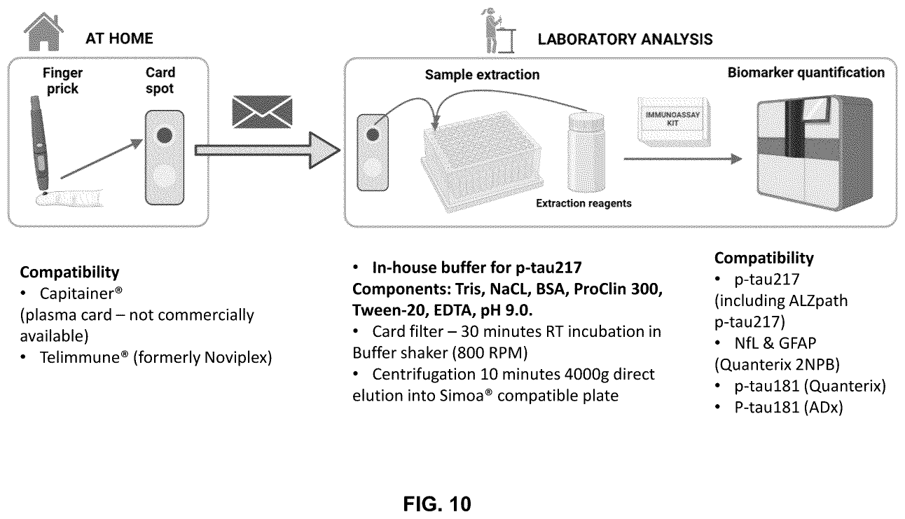

Once the subject has received the card or is ready for sample collection, the subject can use a suitable lancet to prick his or her finger and apply a single hanging drop of blood to each card. The subject should allow the blood drops to dry completely (e.g., for approximately 10 minutes) before sending the cards to the laboratory for analysis using regular post. FIG. 10 shows an overview of the suggested workflow. Briefly, blood collection can be performed at home by the subject or another individual. The blood is spotted on the cards and let dry before sending to the laboratory for analysis. DPS cards may be sent through regular post to be delivered at the laboratory for biomarker detection and quantification. Capitainer plasma cards or Telimmune (formerly Noviplex) are compatible with extraction methods and buffers described in the previous examples, and can be used in this example. Once received at the laboratory, analysis is conducted starting with protein extraction. For p-tau217 detection, a protein extraction buffer of Tris, NaCl, BSA, ProClin™ 300, TRU Block™, Tween-20, and EDTA at a pH of 9.0 is used. The sample is then extracted at the laboratory using the methods and buffers described in the previous examples to detect specific markers such as p-tau217, NfL &GFAP, and p-tau181. For handling card filters, a 30 minute room temperature in protein extraction buffer if a buffer shaker (800 RPM) is conducted. This is followed by centrifugation for 10 minutes at 4000 g for direct elution using a selected elution solution (e.g., deionized water, ultrapure water, or TE) into a Simoa® compatible plate. These samples do not require transfer to a new plate for analysis via Simoa®. Antibodies specific for the selected markers are used in this immunoassay. For biomarker detection and quantification, this method is compatible with the following antibodies: p-tau217 (ALZpath), NfL and GFAP (Quanterix), p-tau181 (Quanterix), and p-tau181 (ADx). Quantified levels of assayed biomarkers are determined and compared to a threshold value derived from a control group. If the level of one or more biomarkers is greater than the threshold value, the subject is determined to have, or be at elevated risk for developing, a neurological condition. For instance, an elevated p-tau217 level in a capillary sample derived from a subject compared to a threshold level of capillary p-tau217 determined from analysis of amyloid negative control subjects indicates that the subject with a significantly elevated capillary p-tau217 level has, or is at elevated risk for developing, Alzheimer's Disease. The subject having received a diagnosis for a neurodegenerative condition (e.g., Alzheimer's Disease), or a substantial brain injury (e.g., Acute TBI) is then selected to receive a treatment. A treatment for Alzheimer's Disease may comprise administering an anti-amyloid beta therapy (e.g., Aducanumab, Donanemab, Lecanemab, or Remternetug) to the subject receiving the diagnosis.

The stability of biomarkers on the dried blood drop card is tested. The results indicate that the biomarkers tested are stable for at least 2 weeks at ambient temperatures when stored on the dried blood drop card prior to extraction. Once the laboratory receives the dried blood drop card, the card can be stored at +4° C. for up to 2 months.

### Protocol for Remote Capillary Blood Sample Collect, Laboratory Testing for Biomarker Quantification, and Diagnosis:

The subject receives a dried blood spot microsampling card (e.g., Telimmune™ Plasma Prep Duo Card or Capitainer*B50 dried blood spot microsampling card) or is ready for sample collection. The subject uses a suitable lancet (e.g., a single-use safety lancet (Unistik 3, 30G)) to prick their finger and apply a single hanging drop (˜50-60 μL) of blood to each blood spot collection location on the card. Each dried blood spot microsampling card is used to collect two separate blood spots from the subject which may be processed under identical or distinct extraction protocols. The card is left to dry at room temperature for 15 min. The collected blood drops should be allowed to dry completely prior to sending the microsampling card via regular post to the laboratory for analysis. The biomarkers tested are stable for at least 2 weeks. Once the laboratory receives the cards, they may be stored at +4° C. for up to 2 months.

To Proceed with Biomarker Quantification:
1) Extract the filters from the dried blood drop cards using one of the protein extraction buffer reagents described herein and place the filters into the designated extraction devices (96-well membrane embedded format (Waters: 186002448).
2) Next, 150 μL of the extraction buffer (For pTau biomarkers: 20 mM Tris, 137 mM NaCl, 1% BSA, 10 μg/mL TRU Block™, 0.1% Tween 20, 5 mM EDTA, pH 9.0) are added to each well within the extraction device, and the samples are incubated for 30 minutes at 37° C. shaking at 800 rpm.
   For testing of biomarkers: i) glial fibrillary acidic protein (GFAP), ii) neurofilament light chain (NFL), iii) 42-amino acid β amyloid (Aβ42) peptide, or iv) 40-amino acid β amyloid (Aβ40) peptide, use one of these extraction buffers:
      Phosphate, NaCl, KCl, BSA, TRU Block™, Tween 20, ProClin™ 300, and EDTA, pH 7.4.
      Phosphate, NaCl, KCl, BSA, MgCl2, Dextrose, Urea, 1.0% TRU Block™, ProClin™ 300, pH 7.4
      Advantage diluent (Quanterix)
      Tau 2.0 (Quanterix)
3) Following the incubation period, the extraction devices, along with the biomarker quantification plate, are centrifuged so the sample is directly deposited in the quantification plate, ready to be measured.
4) Move the quantification plate into a selected detection device apparatus and proceed to follow device protocol to obtain a protein quantitation of one or more selected biomarkers.
   a. This methodology has been proved compatible with the following assays
      i. p-tau217 (ALZpath, Simoa platform)
      ii. NfL & GFAP (Quanterix 2NPB, Simoa platform)
      iii. p-tau181 (Quanterix, Platform)
      iv. p-tau181 (ADx, Platform)
   b. This methodology may be compatible with:
      i. Meso Scale Discovery (MSD)
      ii. Lumipulse (Fujirebio)
      iii. Elecsys (Roche)
      iv. Mass Spectrometry
5) Obtain protein quantitation of one or more selected biomarker and compare obtained value from the subject sample to a threshold value.
6) If measured protein level in the subject sample is determined to be above the threshold value for normal control, the subject is diagnosed according to the information ascribed to the particular biomarker or biomarkers (e.g., elevated capillary p-tau217 level indicates presence of Alzheimer's Disease or an elevated risk for developing Alzheimer's Disease).

### Example 6: Ultrasensitive Detection and Quantification of Neuro Biomarkers for Use in Diagnosis and Treatment of Subjects Having a Neurodegenerative Condition or Traumatic Brain Injury Condition

In this example, biomarker correlations obtained from capillary blood samples extracted from dry drop blood using different devices were compared with venous blood samples extracted from dry drop blood. Neuro biomarkers tested include p-tau217 (ALZpath), p-tau217+(Janssen), p-tau217 University of Gothenburg (UGOT)), p-tau181 (UGOT), p-tau181 (Quanterix), p-tau231 (UGOT), Simoa 2-plex Quanterix (NfL & GFAP), and Simoa 4-plex-B Quanterix (NfL, GFAP, t-tau & UCHL1), and BD-tau. In this example, the Simoa® platform was used for this ultra-sensitivity immunoassay.

The protein extraction buffer composition 1 used with the DPS microsampling cards for detecting and quantifying various tau protein neuro biomarker analytes (including p-tau 217, p-tau 217+, p-tau181, p-tau231, and BD-tau was as follows: 0.01M Tris (Trizma® base; 121.14 molecular weight (MW) in g/mol), 0.15M NaCl (MW: 58.44 g/mol), 2% BSA, 0.10% Tween-20, 5 mM EDTA.

Either protein extraction buffer composition 1 or protein extraction buffer composition 2 was used with the DPS microsampling cards for detecting and quantifying other neuro biomarker analytes including NfL, GFAP, total tau (t-tau), and UCHL1. Protein extraction buffer composition 2 was as follows: Phosphate buffer, NaCl, KCl, BSA, TRU Block™, Tween 20, ProClin™ 300, and EDTA, pH 7.4.

### Extraction Method:

Each dried blood spot microsampling card was used to collect two separate blood spots obtained from the subject which were either processed under identical or distinct extraction protocols. If p-tau neuro biomarkers were being tested, the protein extraction buffer composition 1 described above was used. If neuro biomarkers other than p-tau or BD-tau were being tested, the protein extraction buffer composition 2 described above was used.

Following blood spot application from either venipuncture or a capillary blood sample collected using a suitable lancet (e.g., a single-use safety lancet (Unistik 3, 30G)) to prick the finger of the subject and apply a single hanging drop (˜50-60 μL) to the DPS card, the card was left to dry at room temperature for 15 min. Dried blood spot microsampling cards used were as follows: Telimmune™ Plasma Prep Duo Card, Capitainer*B50 dried blood spot microsampling card, or Capitainer® SEP10 dried blood spot microsampling card. Once the laboratory received the cards, they were either processed soon afterwards or stored at +4° C. for up to 2 months.

Capitainer® SEP10, Capitainer® B50 or Telliumme® filter papers were extracted from the device, either manually or in an automated fashion (Capitainer® SEP10). Filters were placed in a precipitation plate (Waters, P/N 186002448) attached to a 96-well conical Simoa plate (Quanterix, 249944). A total of 170 μL of protein extraction buffer composition 1 was added for tau analysis and 300 μl of either protein extraction buffer composition 1 or protein extraction buffer composition 2 for NfL and GFAP analysis. The plate was sealed with a foil cover and incubated at 37° C./500 rpm for 30 minutes. The plate was centrifuged at 2626 g for 15 minutes at room temperature in a plate centrifuge. The flowthrough was collected in the 96-well conical Simoa® plate after centrifugation and was available immediately for measurement. This protocol does not require the collected flowthrough sample to be transferred to a new plate (unless desired for a different type of analysis).

### Biomarker Quantification:

The Simoa® detection platform was used to quantify the selected neuro biomarkers.

Specific biomarkers may also be assayed using detection platforms other than Simoa® such as: MSD, Lumipulse G1200, Mass Spectrometry, Elecsys Cobas e801, Ella, ARCHITECT, Atellica, ARGO p-tau217 Alamar, ARGO p-tau181 Alamar, ARGO p-tau231 Alamar, p-tau212.

Correlation of Capillary Blood Sample Derived Neuro Biomarker Levels to Levels from Other Biological Samples:

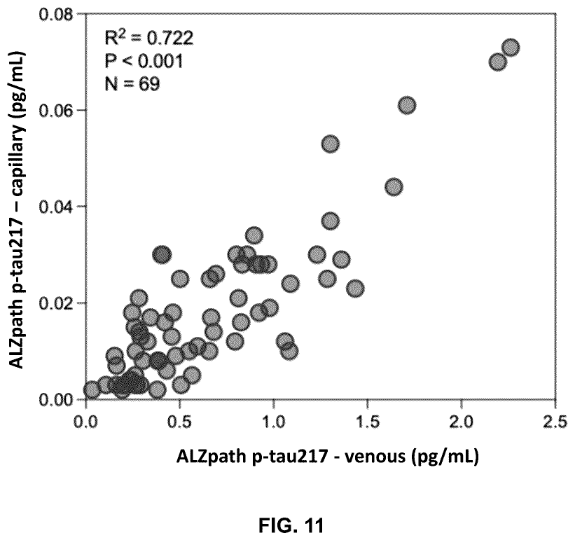

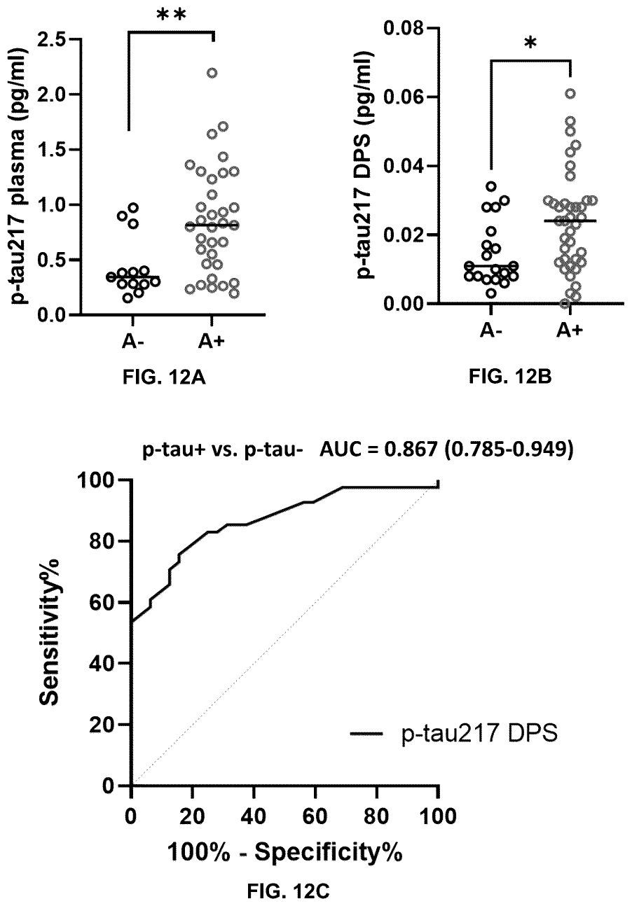

A significant correlation between venous p-tau217 (collected by venipuncture) and capillary p-tau217 was established. In FIG. 11-FIG. 12C, the Simoa® platform was operated using an anti-pTau217 antibody (ALZpath) for immunodetection and quantification. In FIG. 11, p-tau217 levels from capillary blood samples were quantified and correlated to p-tau217 levels from capillary blood samples. This correlation was significant (R²=0.722; p<0.001; N=69) indicating that quantified capillary p-tau217 levels significantly correlate to quantified venous p-tau217 levels (a known neuro biomarker with elevated venous levels indicating presence, or increased likelihood of developing, Alzheimer's Disease). An ALZpath monoclonal antibody specific for p-tau217 was used for this immunoassay. This data used both Capitainer® SEP10 and Telliumme® in 135 individuals from four collection centers for this example. A significant increase of capillary p-tau217 in Aβ-positive individuals and a high predictability of venous p-tau217 using determined cut-points was established. In FIG. 12A, p-tau217 levels were quantified in Aβ-negative individuals (A−) and in Aβ-positive individuals (A+) from plasma samples and assayed using Simoa®. Aβ-positive individuals had a significantly higher p-tau217 level than the control group (p<0.01) (average value of A− is approximately 0.4 μg/mL; average value of A+ is approximately 0.8 μg/mL). In FIG. 12B, p-tau217 levels were quantified in Aβ-negative individuals (A−) and in Aβ-positive individuals (A+) from capillary DPS card samples and assayed using Simoa®. Aβ-positive individuals had a significantly higher p-tau217 level than the control group (p<0.05) (average value of A− is approximately 0.01 μg/mL; average value of A+ is approximately 0.0025 μg/mL). In FIG. 12C, a receiver operating characteristic (ROC) graph is presented as analysis of the Simoa® platform performance for p-tau217 neuro biomarker specificity and sensitivity as described in this example on capillary blood samples collected with DPS cards. Area under the curve (AUC) value was 0.867; confidence interval (0.795-0.949) indicating both high levels of sensitivity and specificity for detected p-tau217 levels in capillary blood samples collected from the subjects.

Additional methods used to determine the diagnostic accuracy of plasma p-tau217 immunoassay for Alzheimer's Disease pathology are described in: Ashton N J, et al. Diagnostic Accuracy of a Plasma Phosphorylated Tau 217Immunoassay for Alzheimer Disease Pathology. JAMA Neurol. 2024 Mar. 1; 81(3):255-263, which is hereby incorporated by reference for methods of sample preparation, p-tau217 detection and quantification, and subsequent diagnosis of an extent of AD pathology.

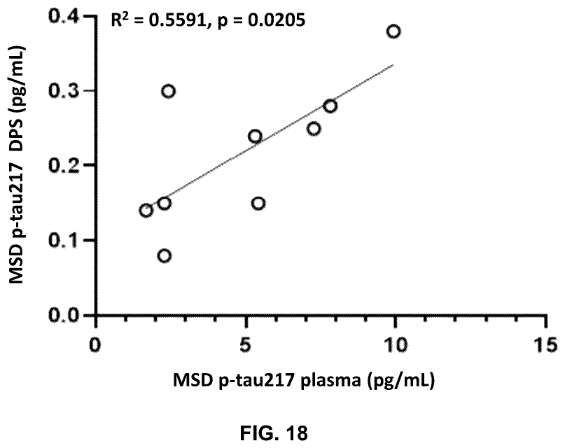

As shown in FIG. 18, ultra-sensitive detection was performed using the Meso Scale Discovery (MSD) platform to investigate the utility of this system for use in detecting and quantifying protein levels of p-tau217 using an p-tau217 antibody. A significant correlation between plasma p-tau217 (collected by venipuncture) and capillary p-tau217 (collected on DPS card) was established. The correlation of established by assaying capillary blood samples for p-tau217 protein levels and comparing to plasma p-tau217 levels confirm that MDS may be used for ultrasensitive quantification of p-tau217 levels from capillary blood samples. From the graph in FIG. 18, R²=0.5591, p=0.0205. Y-axis: capillary p-tau217 on DPS cards (pg/mL); X-axis: plasma p-tau217 (pg/mL).

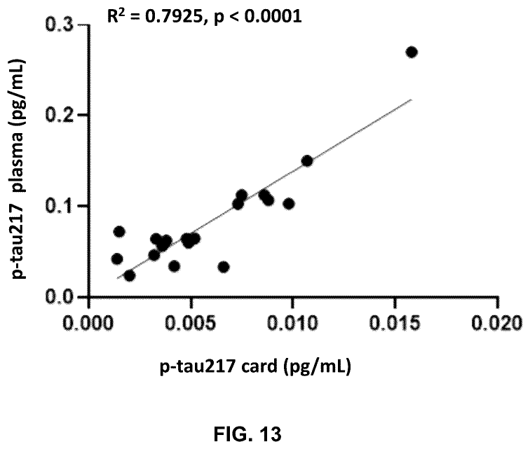

As shown in FIG. 13, Simoa® data generated using the Janssen p-tau217+ antibody and Capitainer® SEP10 DPS card demonstrated an equally strong relationship between capillary blood and venous blood as for the ALZpath p-tau217 assay described above in this example (R²=0.7925; p<0.0001). FIG. 13, shows a graph and significant correlation of capillary p-tau217+ levels compared to venous p-tau217+ levels using Simoa® detection and a Janssen p-tau217+ antibody (venous plasma sample on Y-axis; capillary DPS card collected sample on X-axis; both values listed in pg/mL).

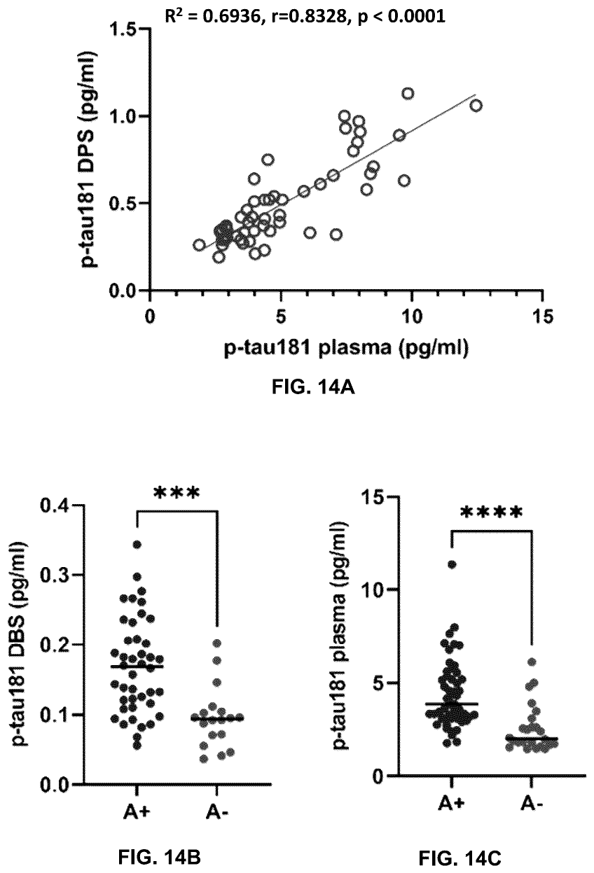

As shown in FIG. 14A-FIG. 14C, Simoa® data generated using the Quanterix p-tau181 antibody (with Capitainer® SEP10 or Telliumme® collection DPS cards) demonstrated a strong relationship between capillary blood and venous blood. FIG. 14A, shows a graph and significant correlation of capillary p-tau181 levels compared to venous p-tau181 levels (R²=0.6936; r-0.8328; p<0.0001) (Y-axis: capillary DPS sample; X-axis: venous plasma sample; both values listed in pg/mL). Significant differences between samples from Ap-positive individuals (A+) compared to Ap-negative controls (A−) were seen for capillary DPS card samples (in FIG. 14B; p<0.001) and for venous plasma samples (in FIG. 14C, p<0.0001). This strong relationship between capillary blood and venous blood offered an equivalent diagnostic as for venous blood to identify Ap-positive individuals.

Simoa® UGOT p-tau181, p-tau217, p-tau231:

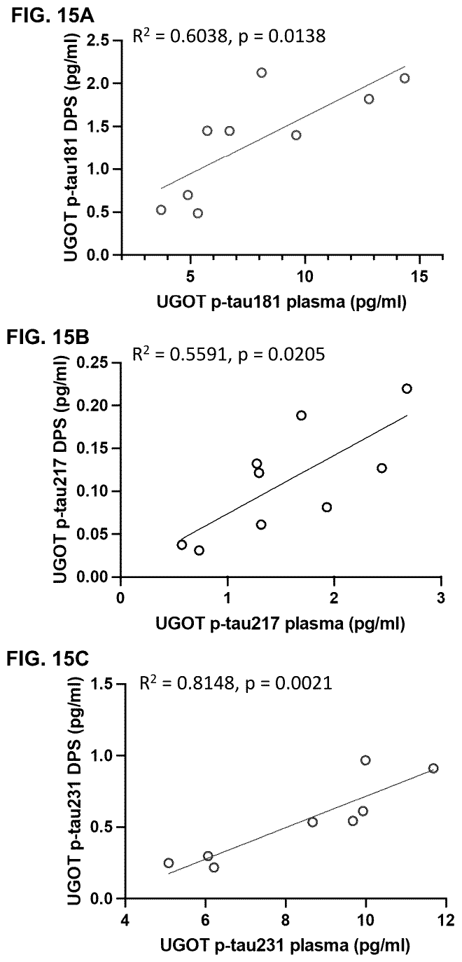

Data generated using the Simoa® UGOT p-tau181 (A), p-tau217 (B) and p-tau231 (C) on Capitainer® SEP10 show significant correlations with venous blood. FIG. 15A shows a graph and significant correlation of capillary p-tau181 DPS levels compared to venous plasma p-tau181 levels (R²=0.6038; p=0.0138) (Y-axis: capillary DPS sample—UGOT p-tau181; X-axis: venous plasma sample—UGOT p-tau181; both values listed in pg/mL). FIG. 15B shows a graph and significant correlation of capillary p-tau217 DPS levels compared to venous plasma p-tau217 levels (R²=0.5591; p=0.0205) (Y-axis: capillary DPS sample—UGOT p-tau217; X-axis: venous plasma sample—UGOT p-tau217; both values listed in pg/mL). FIG. 15C shows a graph and significant correlation of capillary p-tau231 DPS levels compared to venous plasma p-tau231 levels (R²=0.8148; p=0.0021) (Y-axis: capillary DPS sample—UGOT p-tau231; X-axis: venous plasma sample—UGOT p-tau231; both values listed in pg/mL).

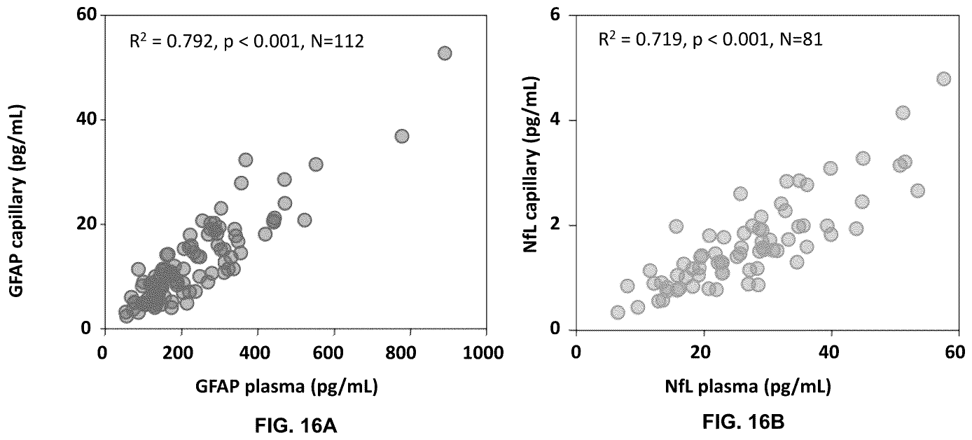

As shown in FIG. 16A-FIG. 16B, Simoa® data generated using a Quanterix Neurofilament light (NfL) antibody and a Quanterix glial fibrillary acidic protein (GFAP) antibody demonstrated a strong relationship between capillary blood and venous blood. FIG. 16A shows a graph and significant correlation of capillary GFAP levels compared to venous GFAP levels (R²=0.792; p<0.001; N=112) (Y-axis: capillary DPS sample; X-axis: venous plasma sample; both values listed in pg/mL). FIG. 16B shows a graph and significant correlation of capillary NfL levels compared to venous NfL levels (R²=0.719; p<0.001; N=81) (Y-axis: capillary DPS sample; X-axis: venous plasma sample; both values listed in pg/mL). These strong relationships between capillary blood and venous blood for levels of GFAP and NfL offer an equivalent diagnostic using capillary blood samples to known neuro biomarkers for neural injury or neurodegenerative disorders (e.g., venous GFAP and venous NfL).

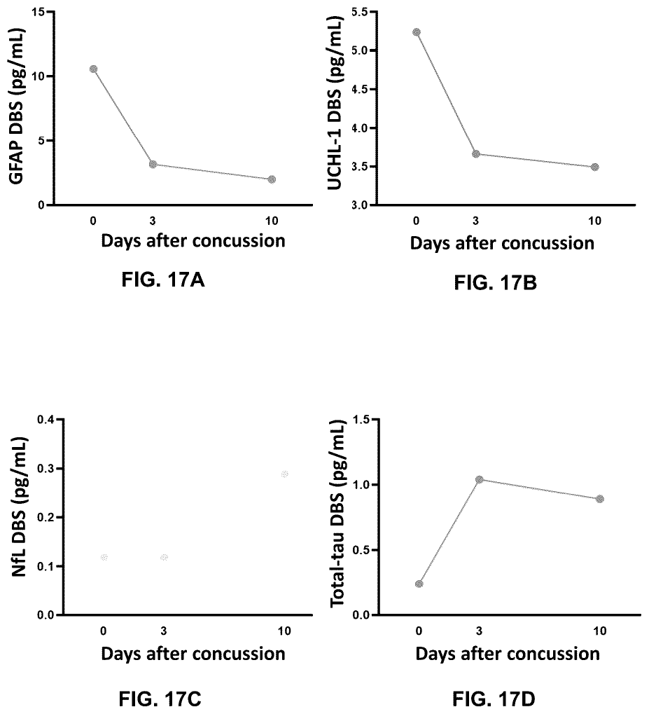

To assess the methods from this example for the application of DPS biomarker changes after concussion, a single sports person had blood samples obtained at three time points for biomarker quantification. Blood collection time points were measured in Days after concussion (Day 0, Day 3, and Day 10). Finger-prick samples of capillary blood were collected using Capitainer® B50 DPS cards. Following protein extraction using the methods described above in this example, four biomarkers (GFAP, UCHL1, NfL, t-tau) were measured using Simoa® detection and quantification to demonstrate the potential to monitor the effects of acute neural injury. As shown in FIG. 17A, levels of GFAP were elevated at Day 0 and were reduced on Day 3 and Day 10 (going from ˜10 μg/mL on Day 0 to ˜2 μg/mL on Day 10). As shown in FIG. 17B, levels of UCHL1 were elevated at Day 0 and were reduced on Day 3 and Day 10 (going from ˜5.25 μg/mL on Day 0 to ˜3.5 μg/mL on Day 3 and Day 10). As shown in FIG. 17C, levels of NfL remained low and within a range of normal at Day 0 and Day 3 (˜0.12 μg/mL). The level of NfL on Day 10 was substantially elevated (˜0.3 μg/mL). As shown in FIG. 17D, levels of total-tau remained low and within a range of normal at Day 0 (˜0.2 μg/mL). The level of total-tau on Day 3 and Day 10 was substantially elevated (˜1.0 μg/mL on Day 3 and ˜0.9 μg/mL on Day 10). These results demonstrate that the effect of acute TBI (e.g., a concussion) can be seen a specific elevations and changes in biomarker levels from Day 0 onward detected from capillary blood samples obtained from a subject and spotted on DPS cards prior to protein extraction. GFAP and UCHL-1 levels were elevated on Day 0 and both were reduced by Day 3 and maintained that level of reduction on Day 10. Total tau (t-tau) was normal on Day 0, but elevated by Day 3 and remained elevated at Day 10. NfL was normal on Day 0 and on Day 3, but was found to be elevated by Day 10.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for determining protein levels of one or more biomarkers in a biological sample, the method comprising:
a) obtaining the biological sample, wherein the biological sample is a capillary blood sample derived from a subject;
b) extracting proteins from the biological sample using a protein extraction reagent to obtain a protein-enriched extracted sample;
c) performing an assay on the protein-enriched extracted sample, wherein the assay comprises determining a detected protein level of at least one of biomarkers: i) phosphorylated Tau-181 (pTau-181), ii) phosphorylated Tau-217 (pTau-217), iii) glial fibrillary acidic protein (GFAP), iv) neurofilament light chain (NFL), v) phosphorylated Tau-231 (pTau-231), vi) phosphorylated Tau-212 (pTau-212), vii) phosphorylated Tau-212+217 (pTau-217+), viii) brain-derived tau (BD-Tau), ix) 42-amino acid 3 amyloid (Aβ42) peptide, or x) 40-amino acid 3 amyloid (Aβ40) peptide.

2. The method of claim 1, wherein the protein extraction reagent comprises a buffered solution comprising tris(hydroxymethyl)aminomethane (Tris), sodium chloride (NaCl), bovine serum albumin (BSA), and a polysorbate nonionic surfactant.

3. The method of claim 1, wherein the protein extraction reagent comprises a buffered solution comprising phosphate buffered saline (PBS) comprising phosphate, NaCl, and potassium chloride (KCl).

4. The method of claim 3, wherein the buffered solution further comprises a polysorbate nonionic surfactant.

5. The method of any one of claims 2-4, wherein the buffered solution further comprises EDTA.

6. The method of any one of claims 3-5, wherein the buffered solution further comprises BSA.

7. The method of any one of claims 3-6, wherein the buffered solution further comprises magnesium chloride (MgCl₂), dextrose, and urea.

8. The method of any one of claims 3-7, wherein the buffered solution further comprises a TRU Block™ immunoassay blocker.

9. The method of any one of claims 2-7, wherein the protein extraction reagent further comprises a preservative.

10. The method of claim 9, where the preservative comprises ProClin™ 300.

11. The method of any one of claims 2-10, wherein the polysorbate nonionic surfactant comprises either polyethylene glycol sorbitan monolaurate (Tween 20) or polyethylene glycol sorbitan monooleate (Tween 80).

12. The method of any one of claims 2-11, wherein the polysorbate nonionic surfactant comprises Tween 20.

13. The method of any one of claims 2-12, wherein the buffered solution has a pH between about 7.0-9.0.

14. The method of any one of claims 3-13, wherein the buffered solution has a pH of about 7.4.

15. The method of any one of claims 2-13, wherein the buffered solution has a pH of about 9.0.

16. The method of any one of claims 8-15, wherein the TRU Block™ immunoassay blocker of the buffered solution is at concentration of about 1%.

17. The method of any one of claims 2, 5, 9-13, or 15 wherein the protein extraction reagent comprises about 20 mM Tris, about 137 mM NaCl, about 1% BSA, about 0.05% ProClin™ 300, about 0.1% Tween 20, and about 5 mM EDTA.

18. The method of any one of claims 1-17, wherein the step b) further comprises incubating the biological samples for 30 minutes.

19. The method of any one of claims 1-18, wherein the step b) further comprises transferring the biological sample to a quantification plate.

20. The method of claim 19, wherein the biological sample is transferred to the quantification plate via centrifugation through a filter plate.

21. The method of any one of claims 1-20, wherein the assay in step c) comprises an immunoassay.

22. The method of claim 21, wherein the immunoassay comprises Western blot analysis, dot blot analysis, flow cytometry-based immunoassay, enzyme-linked immunosorbent assay (ELISA), lateral flow immunoassay, radioimmunoassay (RIA), competition immunoassay, dual antibody sandwich assay, chemiluminescent assay, bioluminescent assay, fluorescent assay, or agglutination assay.

23. The method of claim 21 or 22, wherein the immunoassay comprises: i) single-molecule immunosorbent assay (Simoa®), ii) Meso Scale Discovery high performance electrochemiluminescence (MSD) assay, iii) Lumipulse® assay, iv) Elecsys® assay, v) Ella® Immunoassay (ProteinSimple, Bio-Techne), vi) ARCHITECT® Immunoassay (Abbott), vii) Atellica® Immunoassay (Siemens Healthineers), viii) ARGO™ HT Immunoassay (Alamar), or ix) Olink® immunoassay platform proximity extension assay system (Thermo Fisher Scientific).

24. The method of any one of claims 21-23, wherein the immunoassay comprises i) Simoa®.

25. The method of any one of claims 1-20, wherein the assay comprises mass spectrometry.

26. The method of any one of claims 1-25, wherein the capillary blood sample comprises a plurality of drops of blood.

27. The method of any one of claims 1-25, wherein the capillary blood sample comprises a single drop of blood.

28. The method of any one of claims 1-27, wherein the capillary blood sample has been deposited on a blood sample collection card to create a spotted blood sample.

29. The method of any one of claims 1-28, wherein the volume of the spotted blood sample is less than about 100 μL.

30. The method of claim 28 or 29, wherein the blood sample collection card comprises a plasma separation card.

31. The method of claim 30, wherein the plasma separation card generates cell-free plasma from the spotted blood sample to use as the biological sample in step a).

32. The method of claim 31, wherein cell-free plasma is collected on at least two plasma separation membranes.

33. The method of claim 32, wherein at least one of the at least two plasma separation membrane undergoes protein extraction in step b) using a protein extraction reagent comprising a buffered solution comprising Tris, NaCl, BSA, and a polysorbate nonionic surfactant; wherein at least one of biomarkers: i) pTau-181, ii) pTau-217, v) pTau-231, vi) pTau-212, vii) pTau-217+, or viii) BD-Tau is assayed to determine the detected protein level.

34. The method of claim 32 or 33, wherein at least one of the at least two plasma separation membrane undergoes protein extraction in step b) using a protein extraction reagent comprising a buffered solution comprising Tris, NaCl, BSA, and a polysorbate nonionic surfactant; wherein at least one of biomarkers: i) pTau-181, ii) pTau-217, or v) pTau-231 is assayed to determine the detected protein level.

35. The method of claim 33 or 34, wherein ii) pTau-217 is assayed to determine the detected protein level.

36. The method of claim 33, wherein at least two of i) pTau-181, ii) pTau-217, v) pTau-231, vi) pTau-212, vii) pTau-217+, or viii) BD-Tau are assayed to determine the detected protein level.

37. The method of any one of claims 32-36, wherein the protein extraction reagent further comprises EDTA.

38. The method of any one of claims 32-37, wherein at least one of the at least two plasma separation membrane undergoes protein extraction in step b) using a protein extraction reagent comprising a buffered solution comprising PBS; wherein at least one of biomarkers: iii) GFAP, iv) NFL, vi) Aβ42 peptide, or vii) Aβ40 peptide is assayed to determine the detected protein level.

39. The method of any one of claim 1-38, wherein the detected protein level of the biomarkers: ii) phosphorylated Tau-217 (pTau-217), iii) glial fibrillary acidic protein (GFAP), and iv) neurofilament light chain (NFL), are determined in step c).

40. The method of any one of claim 1-39, wherein the detected protein level of the biomarkers: i) phosphorylated Tau-181 (pTau-181), ii) phosphorylated Tau-217 (pTau-217), iii) glial fibrillary acidic protein (GFAP), and iv) neurofilament light chain (NFL), are determined in step c).

41. The method of any one of claims 1-40, wherein the capillary blood sample is dried prior to step b).

42. The method of any one of claims 1-41, wherein the biological sample is stored for up to about two weeks at ambient temperature prior to extracting proteins in step b).

43. The method of any one of claims 1-42, wherein the biological sample is stored for up to about two months at about 4° C. prior to extracting proteins in step b).

44. The method of any one of claims 1-43, wherein the detected protein level of each biomarker tested derived from the capillary blood sample is significantly correlated with a determined protein level of each respective biomarker tested in plasma isolated from a venous blood sample from the subject.

45. The method of any one of claims 1-44, wherein the detected protein level of each biomarker tested derived from the capillary blood sample is significantly correlated with a determined protein level of each respective biomarker tested in a cerebrospinal fluid (CSF) sample from the subject.

46. The method of any one of claims 1-45, wherein the detected protein level of each biomarker tested is used to assess a risk of the subject for developing cognitive decline as a consequence of neurodegeneration, traumatic brain injury (regardless of severity, including mild traumatic brain injury or concussion), cardiac arrest (or other causes of brain hypoxia), a neuroinflammatory condition, or a combination thereof.

47. The method of any one of claims 1-46, wherein the detected protein level of each biomarker tested is used to pre-screen and identify if the subject has elevated risk for developing cognitive decline prior to the subject undergoing a clinical evaluation.

48. The method of claim 46 or 47, wherein a risk of the subject developing cognitive decline is determined prior to the subject presenting one or more behavioral symptoms associated with cognitive decline.

49. The method of any one of claims 1-48, wherein the detected protein level of each biomarker tested is used as a component in diagnosis of a neurodegenerative condition.

50. The method of any one of claims 1-48, wherein the detected protein level of each biomarker tested is determined in at least two biological samples, each taken from the subject at different times.

51. The method of claim 50, wherein a change in detected protein level of each biomarker tested over time is used to monitor a progression of a neurodegenerative condition.

52. The method of claim 50 or 51, wherein a change in detected protein level of each biomarker tested over time is used to assess a response to a treatment in the subject for a neurodegenerative condition.

53. The method of claim 50 or 51, wherein a change in detected protein level of each biomarker tested over time is used to assess an occurrence of an adverse effect during a treatment with an amyloid immunotherapy.

54. The method of claim 53, wherein the adverse effect comprises amyloid related imaging abnormalities (ARIA).

55. The method of claim 53 or 54, wherein the method further comprises sequentially monitoring an evolution of the adverse effect or sequentially monitoring a resolution of the adverse effect.

56. The method of any one of claim 49-55, wherein the neurodegenerative condition comprises a tauopathy.

57. The method of claim 56, wherein the tauopathy comprises Alzheimer's disease (AD) or Alzheimer's disease pathology or pathophysiology, preclinical AD, prodromal AD, AD dementia, Pick's disease, Niemann-Pick disease type C, frontotemporal dementia (FTD), frontotemporal lobar degeneration (FLD), chronic traumatic encephalopathy (CTE) or traumatic encephalopathy syndrome (TES), progressive supranuclear palsy (PSP), corticobasal degeneration (CBD), Lytico-Bodig disease, tangle-predominant dementia, meningioaniomatosis, primary age-related tauopathy (PART), Argyrophilic grain disease (AGD), globular glial tauopathy (GGT), vacuolar tauopathy, tuberous sclerosis, postencephalitic parkinsonism, subacute sclerosing panencephalitis, amyotrophic lateral sclerosis, myotonic dystrophy, Pallido-ponto-nigral degeneration, Parkinson's disease, Creutzfeldt-Jacob disease, Dementia pugilistica, Down's syndrome, Gerstmann-Staussler-Scheinker disease, inclusion-body myositis, diffuse neurofibrillary tangles with calcification, Tangle-only dementia, Hallevorden-Spatz disease, or a transgene-induced tauopathy.

58. The method of claim 56 or 57, wherein the tauopathy comprises Alzheimer's disease.

59. A method of identifying if a subject is at risk for having or developing a neurodegenerative disease, the method comprising:
a) obtaining a biological sample, wherein the biological sample is a blood sample derived from the subject;
b) extracting proteins from the biological sample using a protein extraction reagent to obtain a protein-enriched extracted sample;
c) performing an assay on the protein-enriched extracted sample, wherein the assay comprises determining a detected protein level of at least one of biomarkers: i) phosphorylated Tau-181 (pTau-181), ii) phosphorylated Tau-217 (pTau-217), iii) glial fibrillary acidic protein (GFAP), iv) neurofilament light chain (NFL), v) phosphorylated Tau-231 (pTau-231), vi) phosphorylated Tau-212 (pTau-212), vii) phosphorylated Tau-212+217 (pTau-217+), viii) brain-derived tau (BD-Tau), ix) 42-amino acid β amyloid (Aβ42) peptide, or x) 40-amino acid 1 amyloid (Aβ40) peptide.
d) providing the detected levels of biomarkers to perform an evaluation step, wherein the evaluation step comprises comparing the provided detected levels of biomarkers in the protein-enriched extracted sample derived from the subject to threshold levels of biomarkers detected in a plurality of healthy subjects having normal cognitive function who have not been diagnosed with a neurodegenerative disease; and
e) estimating a likelihood of the subject for having a neurodegenerative disease or developing a neurodegenerative disease, following completion of the evaluation step.

60. The method of claim 59, wherein the blood sample is a capillary blood sample.

61. The method of claim 59, wherein the blood sample is a venous blood sample.

62. A method for treating a neurodegenerative disease in a subject in need thereof, the method comprising:
i) determining protein levels of one or more biomarkers in a biological sample by:
a) obtaining a biological sample, wherein the biological sample is a blood sample derived from the subject;
b) extracting proteins from the biological sample using a protein extraction reagent to obtain a protein-enriched extracted sample; and
c) performing an assay on the protein-enriched extracted sample, wherein the assay comprises determining a detected protein level of at least one of biomarkers: i) phosphorylated Tau-181 (pTau-181), ii) phosphorylated Tau-217 (pTau-217), iii) glial fibrillary acidic protein (GFAP), iv) neurofilament light chain (NFL), v) phosphorylated Tau-231 (pTau-231), vi) phosphorylated Tau-212 (pTau-212), vii) phosphorylated Tau-212+217 (pTau-217+), viii) brain-derived tau (BD-Tau), ix) 42-amino acid β amyloid (Aβ42) peptide, or x) 40-amino acid β amyloid (Aβ40) peptide; and
ii) administering a therapy to the subject.

63. The method of claim 62, wherein the blood sample is a capillary blood sample.

64. The method of claim 62, wherein the blood sample is a venous blood sample.

65. A kit comprising a protein extraction reagent and instructions for use, wherein the protein extraction reagent comprises less than about 25 mM Tris base, than less about 175 mM NaCl, less than about 2.5% BSA, and less than about 0.15% of a polysorbate nonionic surfactant.

66. The kit of claim 65, wherein the protein extraction reagent comprises 20 mM Tris base, 137 mM NaCl, 1% BSA, and 0.1% Tween-20.

67. The kit of claim 65, wherein the protein extraction reagent comprises 10 mM Tris base, 150 mM NaCl, 2% BSA, and 0.1% Tween-20.

68. The kit of any one of claims 65-67, wherein the protein extraction reagent further comprises 5 mM EDTA.

69. The kit of any one of claims 65-68, wherein the protein extraction reagent further comprises 0.05% ProClin™ 300.

70. The kit of any one of claims 65-69, wherein the instructions comprise instructions for a method of protein extraction and ultra-sensitive quantification described herein.

71. The kit of any one of claims 65-70, further comprising a quantification plate.

72. The kit of claim 71, wherein the quantification plate is compatible for use in a Simoa® detection system, an Meso Scale Discovery (MDS) analysis system, a Lumipulse® immunoassay analysis system, an Elecsys Cobas® e801 immunohistochemistry analysis system, an Ella Automated Immunoassay System analysis system, an ARCHITECT Immunoassay System analysis system, an Atellica® Solution Immunoassay analysis system, an ARGO™ HT System Immunoassay analysis system, or a mass spectrometer.

73. The kit of any one of claims 65-72, further comprising an antibody specific for detection of one or more selected neuro biomarkers.

74. The kit of claim 73, wherein the antibody specific for detection of one or more selected neuro biomarkers is a p-tau181 antibody, a p-tau212 antibody, a p-tau217 antibody, a p-tau217+ antibody, a p-tau-231 antibody, a total-tau antibody, a BD-tau antibody, a GFAP antibody, an NfL antibody, an Aβ40 antibody, an Aβ42 antibody, or an UCHL1 antibody.
